(19) United States
(12) Patent Application Publication
(10) Pub. No.: US 2026/0183422 A1
(43) Pub. Date: Jul. 2, 2026

(54) COMPOSITIONS FOR TREATING FRIEDREICH'S ATAXIA

(71) Applicant: The Trustees of the University of Pennsylvania, Philadelphia, PA (US)

(72) Inventors: James M. Wilson, Philadelphia, PA (US); Christian Hinderer, Baltimore, MD (US); Nimrod Miller, Berwyn, PA (US)

(73) Assignee: The Trustees of the University of Pennsylvania, Philadelphia, PA (US)

(21) Appl. No.: 19/566,500

(22) Filed: Mar. 13, 2026

Related U.S. Application Data
(63) Continuation of application No. 17/787,013, now Pat. No. 12,594,348.
(60) Provisional application No. 62/950,834, filed on Dec. 19, 2019.

(51) Int. Cl. A61K 48/00; A61K 47/02; A61P 25/28; C12N 15/86

(52) U.S. Cl. CPC A61K 48/0008; A61K 47/02; A61K 48/0075; A61P 25/28; C12N 15/86; C12N 2750/14122; C12N 2750/14143; C12N 2750/14152

(57) ABSTRACT

A recombinant adeno-associated virus (rAAV) comprising an AAV capsid and a vector genome comprising a frataxin gene is provided. Also provided is a composition containing an effective amount of rAAV to ameliorate symptoms of Freidreich's ataxia, including, e.g., reduction in progression towards neurocognitive decline and/or cardiomyopathy.

### CROSS-REFERENCE TO RELATED APPLICATIONS

This is a continuation of U.S. patent application Ser. No. 17/787,013 filed Jun. 17, 2022, which is a National Stage Entry under 35 U.S.C. 371 of International Patent Application No. PCT/US2020/066167, filed Dec. 18, 2020, which claims the benefit of U.S. Provisional Application No. 62/950,834, filed Dec. 19, 2019. These applications are incorporated by reference herein in their entirety.

### REFERENCE TO AN ELECTRONIC SEQUENCE LISTING

The electronic sequence listing filed herewith named “UPN-20-9217USC1.xml” with size of 570,555 bytes, created on date of Mar. 13, 2026, and the contents of the electronic sequence listing (e.g., the sequences and text therein) are incorporated herein by reference in entirety.

### BACKGROUND OF THE INVENTION

Friedreich's ataxia (FRDA) is a rare genetic disorder characterized by progressive neurological symptoms, cardiomyopathy and increased risk of diabetes. Patients typically present in late childhood or early adolescence with ataxia, dysarthria and spasticity [Harding, A. E. Friedreich's ataxia: a clinical and genetic study of 90 families with an analysis of early diagnostic criteria and intrafamilial clustering of clinical features. Brain: a journal of neurology 104, 589-620 (1981)]. Most become wheelchair dependent in the third decade due to worsening ataxia, though lower extremity strength often remains intact [Harding, cited above]. Sensory neuropathy predominately affecting proprioception presents early and shows little progression later in the disease course [Koeppen, A. H. Friedreich's ataxia: pathology, pathogenesis, and molecular genetics. Journal of the neurological sciences 303, 1-12 (2011)]. Most FRDA patients develop a hypertrophic cardiomyopathy which is frequently the cause of death in the fifth or sixth decade. Virtually all FRDA patients exhibit impaired glucose tolerance, and about 10% develop diabetes [Harding, cited above; Koeppen, cited above].

FRDA is caused by recessively inherited mutations in the gene encoding frataxin, a ubiquitous mitochondrial protein involved in iron metabolism. The most common pathogenic allele is a large noncoding trinucleotide repeat expansion in the first intron of the frataxin gene [Cocozza, S., Koenig, M. & Pandolfoll, M. Friedreich's ataxia: autosomal recessive disease caused by an intronic GAA triplet repeat expansion. Science (New York, N.Y.) 271, 1423 (1996)]. The expansion inhibits transcription of the gene, resulting in a 70-80% reduction in frataxin protein [Planese L., et al. Real time PCR quantification of frataxin mRNA in the peripheral blood leucocytes of Friedreich ataxia patients and carriers. Journal of Neurology, Neurosurgery & Psychiatry 75, 1061-1063 (2004)]. Additional loss-of-function mutations have been identified in some FRDA patients, indicating that the repeat expansion causes disease through inhibition of expression rather than a toxic gain-of-function mechanism [Cocozza, cited above]. Residual frataxin protein expression is inversely proportional to repeat length, and patients with longer repeat expansions have earlier symptom onset [Filla, A., et al. The relationship between trinucleotide (GAA) repeat length and clinical features in Friedreich ataxia. American Journal of Human Genetics 59, 554-560 (1996)]. The neurological phenotype of FRDA can be localized to three cell types that are selectively susceptible to frataxin deficiency [Koeppen, cited above; Filla, cited above]. Neurons in the dentate nuclei of the cerebellum demonstrate marked degeneration, which correlates clinically with ataxia, dysmetria and dysarthria. There is also degeneration of upper motor neurons and the corresponding axons in the corticospinal tract, giving rise to spasticity. The sensory neuropathy is caused by death of sensory neurons in the dorsal root ganglia. The central nervous system lesions of FRDA are remarkably specific to these three cell types, with sparing of the rest of the brain and spinal cord in most patients [Koeppen, cited above; Filla, cited above].

Adeno-associated virus (AAV), a member of the Parvovirus family, is a small non-enveloped, icosahedral virus with single-stranded linear DNA (ssDNA) genomes of about 4.7 kilobases (kb) long. The wild-type genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which self-assemble to form a capsid of an icosahedral symmetry.

AAV is assigned to the genus, Dependovirus, because the virus was discovered as a contaminant in purified adenovirus stocks. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer.

What is desirable are alternative therapeutics for treatment of patients having conditions associated with an abnormal FXN gene.

### SUMMARY OF THE INVENTION

A therapeutic, recombinant (r), replication-defective, adeno-associated virus (AAV) is provided which is useful for treating and/or reducing the symptoms associated with Freidreich's ataxia (FA or FRDA) in human patients in need thereof. A recombinant adeno-associated virus (rAAV) is provided which comprises an AAV capsid and a vector genome comprising an FXN gene having the sequence of SEQ ID NO: 3 or a sequence 95% identical thereto that encodes a human frataxin, and regulatory sequences which direct expression of the FXN gene in targeted human cells. In certain embodiments, the FXN gene encodes for an amino acid sequence of SEQ ID NO: 2 or a sequence at least about 95% identical thereto. In certain embodiments, the vector genome comprises an AAV2 5′ inverted terminal repeat (ITR) a CB7 promoter, an intron, the FXN gene, a polyA, and an AAV2 3′ ITR. In certain embodiments, the vector genome comprises See, SEQ ID NO: 8, or See, also, SEQ ID NO: 12. In certain embodiments, the vector genome comprises a 5′ ITR, nucleotides 198 to 2737 of SEQ ID NO: 8 and a 3′ ITR. In the embodiments, the vector genome comprises a 5′ ITR, nucleotides 198 to 2736 of SEQ ID NO: 12, and a 3′ ITR. In some embodiments, the vector genome comprises at least one, at least two, or at least three tandem repeats of dorsal root ganglion (DRG)-specific miRNA targeted sequences. In certain embodiments, the AAV capsid is selected from AAVrh.91 or AAVhu68. In some embodiments, the AAVhu68 capsid comprise a heterogeneous population of vp1 (aa Ito 736 of SEQ ID NO: 5; encoded by nt 1 to 2211 of SEQ ID NO: 4), vp2 (aa 138 to 736 of SEQ ID NO: 5; encoded by nt 412 to 2211 of SEQ ID NO: 4), and vp3 (aa 203 to 736 of SEQ ID NO: 5; encoded by nt 607 to 2211 SEQ ID NO: 4) proteins.

Also provided herein is a pharmaceutical composition comprising a formulation buffer and a stock of a rAAV.FXN as described herein. In certain embodiments, the formulation buffer comprises an artificial cerebrospinal fluid which comprises of a buffered saline and one or more of sodium, calcium, magnesium, potassium, or mixtures thereof, and a surfactant. In some embodiments, the pharmaceutical composition is at a pH in the range of 7.5 to 7.8, or 6.2 to 7.7, or about 7.

Also provided herein is a regimen comprising dual-route of administration of the rAAV or the pharmaceutical composition wherein such regimen comprising of intravenous administration and intraparenchymal (dentate nucleus) administration to a patient in the need thereof. In some embodiments, intravenous administration and intraparenchymal administration are performed sequentially and within 24-hours of each other. In some embodiments, the regimen comprising intravenous and intrathecal administration of the rAAV or the pharmaceutical composition.

In certain embodiments, the rAAV or the pharmaceutical composition provided herein are useful in treatment of FRDA, wherein the treatment comprises of a dual-route administration, and includes amelioration of FRDA by reducing of cardiac and/or neurological symptoms. In some embodiments, the amelioration of FRDA includes increased average life span, reduction in progression towards neuromuscular decline, improvement in neuromuscular development, reduction in progression towards cardiomyopathy and/or improvement in cardiac symptoms. In certain embodiments, the rAAV or the pharmaceutical composition is suitable for intravenous, intraparenchymal and/or intrathecal administration to a patient in the need thereof.

In certain embodiments, a method or a use of a composition is provided for treating FRDA in a subject with FRDA and/or ameliorating one or more symptoms of FRDA, wherein the method or the use of a composition comprising administering of the rAAV or the pharmaceutical composition provided herein to a subject via intravenous, intraparenchymal, and/or intrathecal delivery In certain embodiments, a method or a use of a composition, comprises dual-route administration including intravenous and intraparenchymal administration, wherein the rAAV or the pharmaceutical composition is administered at a ratio of about 20:1 to about 1:1, preferably about 10:1 (intravenous:intraparenchymal). In certain embodiments, the dual route of administration may be used for delivery in combination with an rAAV.FXN and a second active component for treating FA patients.

Also provided herein is a plasmid comprising an expression cassette which comprises a FXN gene having the sequence of SEQ ID NO: 3 or a sequence 95% identical thereto that encodes human frataxin. In certain embodiments, the FXN gene encodes a frataxin protein having the sequence of SEQ ID NO: 2, or a sequence at least 95% identical thereto. In certain embodiments, the plasmid comprises a vector genome which comprises AAV2 5′ ITR, CB7 promoter, an intron, a polyA, and an AAV2 3′ ITR. Host cells comprising the plasmids described herein are also provided. In certain embodiments, the vector genome comprises See, SEQ ID NO: 8, or See, also, SEQ ID NO: 12. In certain embodiments, the vector genome comprises a 5′ ITR, nucleotides 198 to 2737 of SEQ ID NO: 8 and a 3′ ITR. In the embodiments, the vector genome comprises a 5′ ITR, nucleotides 198 to 2736 of SEQ ID NO: 12, and a 3′ ITR.

The activity of a rAAV.hFXN via intravenous administration has been evaluated in a murine model of FRDA cardiomyopathy. rAAV.hFXN administration increased heart frataxin levels and significantly improved survival. Studies in nonhuman primates have demonstrated that the rAAV.hFXN can efficiently express frataxin in key cellular targets with an acceptable safety profile. In certain embodiments, rAAV.hFXN may be suitable for combined central and intravenous routes of administration to address the cardiac and neurological features of FRDA.

These and other aspects of the invention are apparent from the following detailed description of the invention.

### BRIEF DESCRIPTION OF THE DRAWINGS

FIGS. 1A and 1B provide an alignment of wild-type human frataxin (SEQ ID NO: 1) and an engineered human frataxin coding sequence (Human FXN) (SEQ ID NO: 3). FIG. 1A provides an alignment of nucleic acids 1 to 480 of wild-type and an engineered human frataxin coding sequence. FIG. 1B provides an alignment of nucleic acid 481 to 630 of wild-type and an engineered human frataxin coding sequence. The sequences are about 73% identical as determined using an online BLAST tool (blast.ncbi.nlm.nih.gov).

FIGS. 2A and 2B provide the results of a pilot cardiac gene therapy study in a FXN^{-/flox}. Ckmm-Cre^{+/-} FA mouse model. FIG. 2A provides a graph of body weight (grams±SEM) of untreated control mice or mice intravenously administered rAAVhu68.hFXN over time (0-20 weeks). At 30 days of age Fxn cKO mice were IV-administered rAAVhu68.hFXN at a dose of 2.0×10¹¹ GC. Age-matched Fxn cKO mice and Fxn unaffected mice remained untreated and served as controls. Body weights were recorded weekly until human euthanasia criteria were met. Average body weights are presented. Error bars represent the standard error of the mean. Abbreviations: Fxn, frataxin (gene, mouse); GC, genome copies; Fxn cKO, cardiac conditional knockout affected mice (Fxn^{flox/null}::Ckmm-Cre); Fxn unaffected mice (Fxn^{flox/null}); GC, genome copies; IV, intravenous; SEM, standard error of the mean. The triangles represent Fxn^{flox/null}::Ckmm-Cre+rAAVhu68.hFXN (2×10¹¹ genome copies (GC) intravenous (iv) at 5 weeks (n=7). The circles represent results in Fxn^{flox/null}::Ckmm-Cre mice (n=7). The squares represent Fxn^{flox/null} (n=7). FIG. 2B provides percent survival of untreated control mice or mice intravenously administered rAAVhu68.hFXN over time (0 to 20 weeks). At 30 days of age Fxn cKO mice were IV-administered rAAVhu68.hFXN at a dose of 2.0×10¹¹ GC. Age-matched Fxn cKO mice and Fxn unaffected control mice remained untreated and served as controls. Survival was monitored. Abbreviations: Fxn, frataxin (gene, mouse); GC, genome copies; Fxn cKO, cardiac conditional knockout affected mice (Fxn^{flox/null}::Ckmm-Cre); Fxn unaffected control mice (Fxn^{flox/null}).

FIG. 3 provides the GDF-15 levels in the serum following IV administration of rAAVhu68.hFXN in FA mouse model. At 30 days of age Fxn cKO mice were IV-administered rAAVhu68.hFXN at a dose of 2.0×10¹¹ GC. Age-matched Fxn cKO mice and Fxn unaffected control mice remained untreated and served as controls. Blood was collected at necropsy when mice reached the humane euthanasia criteria. **p=0.001 and *p=0.45 based on a statistical analysis using a one-way ANOVA followed by Tukey's. Abbreviations: ANOVA, analysis of variance; Fxn, frataxin (gene, mouse); GC, genome copies; Fxn cKO, cardiac conditional knockout affected mice (Fxn^{flox/null}::Ckmm-Cre); Fxn unaffected control mice (Fxn^{flox/null}).

FIGS. 4A and 4B provide a manufacturing process flow diagram for drug substance. FIG. 4A provides the upstream manufacturing process flow diagram. FIG. 4B provides the downstream manufacturing process flow diagram.

### DETAILED DESCRIPTION OF THE INVENTION

Adeno-associated virus (AAV) based compositions and methods for treating Friedreich's ataxia (FRDA or FA) are provided herein. The rAAV is desirably replication-defective and carries a vector genome comprising a hFXN gene encoding human (h) frataxin under the control of regulatory sequences which direct its expression in targeted human cells; this rAAV may be termed as rAAV.hFXN as used herein. In certain embodiments, the rAAV comprises an AAVhu68 capsid. This rAAV is termed herein rAAVhu68.hFXN, but in certain instances the terms rAAVhu68.HFXN vector, rAAVhu68.hFXN, or AAVhu68.hFXN vector are used interchangeable to reference the same embodiment. In certain embodiments, the vector genome is entirely exogenous to the AAVhu68 capsid, as it contains no AAVhu68 genomic sequences. In certain embodiments, a capsid other than the AAVhu68 capsid may be utilized (e.g. AAVrh91). In a further embodiment, the rAAV has a capsid is suitable for delivering a vector genome into the central nervous system (CNS) and/or intravenously. For example, a vector capsid may target the dentate nuclei in the cerebellum, the cerebellum, the brain, or other cells in the CNS. In certain embodiments, the rAAV compositions Additionally, provided are methods, vectors (viral or non-viral vectors, such as plasmids), and cells for use in production (for example, generation and/or purification) of the rAAV. An effective amount of genome copies (GC) of a recombinant AAV (rAAV) having an AAVhu68 capsid and carrying a vector genome encoding a frataxin (FXN) enzyme (rAAVhu68.FXN) is delivered to the patient. Desirably, this rAAVhu68.FXN is formulated with an aqueous buffer. In certain embodiments, the suspension is suitable for intrathecal injection/infusion, intravenous injection/infusion, or intraparenchymal injection/infusion. In certain embodiments, rAAVhu68.FXN is rAAVhu68.hFXN, in which the FXN gene (i.e., frataxin (also termed as FXN protein) is under the control of regulatory sequences. In certain embodiments, a single route of administration is used for targeting cardiac tissue and/or the central nervous system (e.g., dorsal root ganglia). In certain embodiments, two or more routes of delivery are used. In certain embodiments, one route of delivery is intravenous and the second route of delivery is intraparenchymal.

Reduced expression of frataxin (encoded by the FXN gene) is the cause of Friedreich's ataxia, a neurodegenerative disease. FRDA is characterized by ataxia, sensory loss and cardiomyopathy. With reference to SEQ ID NO: 2, the full-length human frataxin protein is 210 amino acids in length. See, e.g., UniProtKB.org/uniprot/Q16595. The human frataxin protein contains an N-terminal transit peptide (e.g., amino acids 1 to 41, or a fragment thereof). Various forms of frataxin have been described and may have biological function, including a frataxin intermediate form (e.g., about amino acid 20 to about amino acid 210, or about amino acid 42 to about amino acid 210). In certain embodiments, the mature frataxin includes about amino acid 81 to about amino acid 210, which may be sufficient to provide frataxin's biological function. However, in certain embodiments, additional forms of frataxin, e.g., about amino acid 56 to about amino acid 210 of SEQ ID NO: 2, or about amino acids 78 to about amino acid 210 of SEQ ID NO: 2 may provide the desired frataxin biological function. In certain embodiments, more than one form of frataxin is produced following expression of the FXN gene. In certain embodiments, frataxin may be present as a monomer. In certain embodiments, frataxin may be present as an oligomer.

As used herein, “treating Friedrich's ataxia” means to increase expression levels of the human frataxin protein, or a functional form thereof, to a level which improves one or more symptoms of FRDA and/or which prevents progression of the symptoms in a subject. Such symptoms may include one or more of: neurodegeneration and cardiomyopathy, ataxia (impaired ability to coordinate voluntary movements), dysarthria (slurred speech, progressive), spasticity, weakness (progressive), sensory neuropathy, diabetes, nystagmus, diminished or absent tendon reflexes, Babinski sign, impairment of position and vibratory senses, scoliosis (curvature of the spine), pes cavus. and hammer toe. About a third of the people with FRDA develop diabetes mellitus, which usually manifests before adolescence. In some embodiments, onset is between 10 and 15 years and most people are diagnosed before age 25. Late-onset FRDA/very late onset FRDA affect about 15% of FRDA patients. Late-onset FRDA is typically from ages 26 to 39 and very late onset FRDA is typically after 40 years of age. In certain embodiments, treating FRDA includes dual-routes of administration, wherein a composition is administered systemically and to the central nervous system (CNS). In some embodiments, the dual-routes of administration include intravenous (IV) and intraparenchymal (dentate nucleus) (IDN) routes of administration. The dual-routes of administration addresses an unmet need in FRDA patients to stabilize and/or improve the ataxic symptoms of FRDA (CNS) and to prevent cardiac manifestation of FRDA (systemic). Furthermore, the IDN route of administration of a composition addresses neurological manifestations in the dentate nuclei and DRG and treats ataxia, dysmetria, dysarthria along with peripheral neuropathy observed in FRDA patients.

The gene therapy vectors provided herein, i.e., rAAV.FXN (for example, rAAVhu68.FXN), and the compositions comprising the same are useful for treatment of conditions associated with deficiencies in levels of frataxin in a subject. As used herein, a gene therapy vector refers to a rAAV as described herein, which is suitable for use in treating a patient. In certain embodiments, the gene therapy vector or the pharmaceutical composition provided herein is useful for treating FRDA.

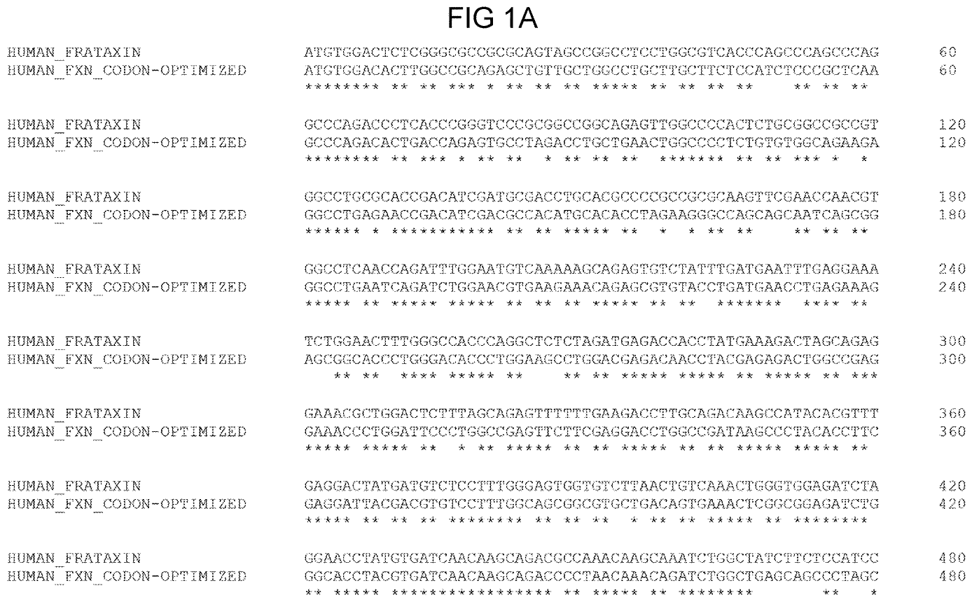

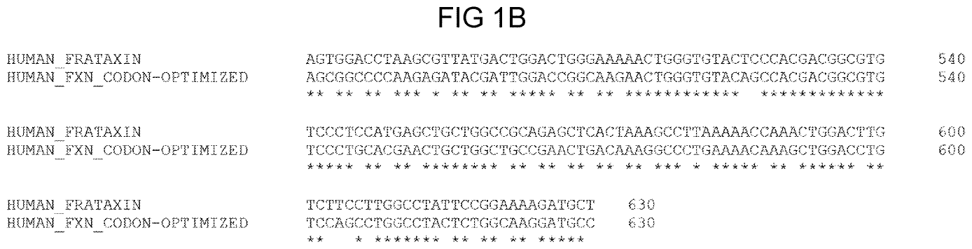

In certain embodiments, an “effective amount” of rAAV.FXN (for example, rAAVhu68.hFXN) as provided herein is the amount which achieves amelioration of one or more symptoms associated with FRDA. The rAAV.FXN described herein, and compositions comprising the same, contain a FXN gene (i.e., frataxin coding sequence) which encodes and expresses human frataxin protein (which may be also termed as FXN enzyme), or a functional fragment thereof. In one embodiment, the FXN gene is engineered to have the sequence of SEQ ID NO: 3, or a sequence at least 95% identical thereto. In certain embodiments, the FXN gene encodes a frataxin protein having an amino acid sequence of SEQ ID NO: 2, or a sequence at least 95% identical thereto. As shown in FIG. 1, SEQ ID NO: 3 is less than 80% identical to the wild-type FXN gene, which is reproduced in SEQ ID NO: 1. As used herein, the term “functional frataxin” refers to an enzyme having the amino acid sequence of the full-length native (wild-type) protein (as shown in SEQ ID NO: 2), a variant thereof, a mutant thereof with a conservative amino acid replacement, a fragment thereof, a full-length or a fragment of any combination of the variant and the mutant with a conservative amino acid replacement, which provides at least about 10%, at least about 20%, at least about 30%, at least about 40%, at least about 50%, at least about 60%, at least about 70%, at least about 75%, at least about 80%, at least about 90%, or about the same, or greater than 100% of the biological activity level of a native (wild-type) frataxin.

In certain embodiments, the native leader sequence of the human FXN gene (e.g., amino acids 1 to 41) may be removed in full or in part and replaced with an exogenous leader sequence. In one embodiment, the leader is from human IL2 or a mutated leader. In another embodiment, a human serpinF1 secretion signal is used as a leader peptide.

The term “expression” is used herein in its broadest meaning and comprises the production of RNA and/or protein. With respect to RNA, the term “expression” or “translation” relates in particular to the production of peptides or proteins. Expression may be transient or may be stable.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As described above, the term “about” when used to modify a numerical value means a variation of ±10%, unless otherwise specified.

As described above, the terms “increase” “decrease” “reduce” “ameliorate” “improve” “delay” “earlier” “slow” “cease” or any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to the corresponding reference (e.g., untreated control, corresponding level of a FA patient or a FA patient at a certain stage or a healthy subject or a healthy human without FA)), unless otherwise specified.

“Patient” or “subject” as used herein refer to a mammalian animal, including a human, a veterinary or farm animal, a domestic animal or pet, and animals normally used for clinical research. In one embodiment, the subject of these methods and compositions is a human. In certain embodiments, the patient has FA.

Unless defined otherwise in this specification, technical and scientific terms used herein have the same meaning as commonly understood by one of ordinary skill in the art and by reference to published texts, which provide one skilled in the art with a general guide to many of the terms used in the present application.

I. rAAV

In certain embodiments, provided herein is a rAAV comprising an AAV capsid and a vector genome packaged therein. The vector genome includes an AAV 5′ inverted terminal repeat (ITR), a nucleic acid sequence encoding a FXN gene as described herein, regulatory sequences which directs expression FXN in a target cell, and an AAV 3′ ITR. Such rAAV are suitable for use in the treatment of Friedreich's ataxia. The gene therapy vectors provided herein, i.e., rAAV.FXN (for example, rAAVhu68.FXN), and the compositions comprising the same are useful for treatment of conditions associated with deficiencies in levels of frataxin in a subject.

As used herein, a “rAAV.hFXN” refers to a rAAV having a vector genome that includes an hFXN coding sequence. A “rAAVhu68.hGLA” refers to rAAV having an hu68 capsid and a vector genome that includes an hFXN coding sequence.

AAVhu68 varies from another Clade F virus AAV9 by two encoded amino acids at positions 67 and 157 of vp1, based on the numbering of SEQ ID NO: 5. In contrast, the other Clade F AAV (AAV9, hu31, hu31) have an Ala at position 67 and an Ala at position 157. AAVhu68 capsids have a valine (Val or V) at position 157 and a glutamic acid (Glu or E) at position 67, based on the numbering of SEQ ID NO: 5. Another suitable capsid is AAVrh91. See WO 2020/223231, published Nov. 5, 2020, U.S. Patent Application No. 63/065,616, filed Aug. 14, 2020, and U.S. Patent Application No. 63/109,734, filed Nov. 4, 2020, which are incorporated herein by reference.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78 (10): 6381-6388, which identifies Clades A, B, C, D, E and F, and provides nucleic acid sequences of novel AAV, GenBank Accession Numbers AY530553 to AY530629. See, also, WO 2005/033321.

AAVhu68 is described in WO 2018/160582, which is incorporated by reference in its entirety herein, and in this detailed description. In certain embodiments, an AAVhu68 capsid is further characterized by one or more of the following: AAVhu68 vp1 proteins produced by expression from a nucleic acid sequence which encodes the predicted amino acid sequence of 1 to 736 of SEQ ID NO: 5, vp1 proteins produced from SEQ ID NO: 4, or vp1 proteins produced from a nucleic acid sequence at least 70% identical to SEQ ID NO: 4 which encodes the predicted amino acid sequence of 1 to 736 of SEQ ID NO: 5; AAVhu68 vp2 proteins produced by expression from a nucleic acid sequence which encodes the predicted amino acid sequence of at least about amino acids 138 to 736 of SEQ ID NO: 5, vp2 proteins produced from a sequence comprising at least nucleotides 412 to 2211 of SEQ ID NO: 4, or vp2 proteins produced from a nucleic acid sequence at least 70% identical to at least nucleotides 412 to 2211 of SEQ ID NO: 4 which encodes the predicted amino acid sequence of at least about amino acids 138 to 736 of SEQ ID NO: 5; and/or AAVhu68 vp3 proteins produced by expression from a nucleic acid sequence which encodes the predicted amino acid sequence of at least about amino acids 203 to 736 of SEQ ID NO: 5, vp3 proteins produced from a sequence comprising at least nucleotides 607 to 2211 of SEQ ID NO: 4, or vp3 proteins produced from a nucleic acid sequence at least 70% identical to at least nucleotides 607 to 2211 of SEQ ID NO: 4 which encodes the predicted amino acid sequence of at least about amino acids 203 to 736 of SEQ ID NO: 5.

The AAVhu68 vp1, vp2 and vp3 proteins are typically expressed as alternative splice variants encoded by the same nucleic acid sequence which encodes the full-length vp1 amino acid sequence (amino acid (aa) 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence (about aa 203 to 736) without the vp1-unique region (about aa 1 to about aa 137) and/or vp2-unique regions (about aa 1 to about aa 202), or a strand complementary thereto, the corresponding mRNA or tRNA (for example, the mRNA transcribed from about nucleotide (nt) 607 to about nt 2211 of SEQ ID NO: 4), or a sequence at least 70% to at least 99% (e.g., at least 85%, at least 90%, at least 95%, at least 97%, at least 98% or at least 99%) identical to SEQ ID NO: 4 which encodes aa 203 to 736 of SEQ ID NO: 5. Additionally, or alternatively, the vp1-encoding and/or the vp2-encoding sequence may be co-expressed with the nucleic acid sequence which encodes the AAVhu68 vp2 amino acid sequence of SEQ ID NO: 5 (about aa 138 to 736) without the vp1-unique region (about aa 1 to about 137), or a strand complementary thereto, the corresponding mRNA or tRNA (for example, the mRNA transcribed from nt 412 to 2211 of SEQ ID NO: 4), or a sequence at least 70% to at least 99% (e.g., at least 85%, at least 90%, at least 95%, at least 97%, at least 98% or at least 99%) identical to SEQ ID NO: 4 which encodes about aa 138 to 736 of SEQ ID NO: 5.

As described herein, a rAAVhu68 has a rAAVhu68 capsid produced in a production system expressing capsids from an AAVhu68 nucleic acid sequence which encodes the vp1 amino acid sequence of SEQ ID NO: 5, and optionally additional nucleic acid sequences, e.g., encoding a vp3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 5. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 4, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 5 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 4). In certain embodiments, also provided is a nucleic acid sequence which encodes the AAVhu68 vp2 amino acid sequence of SEQ ID NO: 5 (about aa 138 to 736) without the vp1-unique region (about aa 1 to about 137), or a strand complementary thereto, the corresponding mRNA or tRNA (nt 412 to 2211 of SEQ ID NO: 4).

However, other nucleic acid sequences which encode the amino acid sequence of SEQ ID NO: 5 may be selected for use in producing rAAVhu68 capsids. In certain embodiments, the nucleic acid sequence has the nucleic acid sequence of SEQ ID NO: 4 or a sequence at least 70% to 99% identical, at least 75%, at least 80%, at least 85%, at least 90%, at least 95%, at least 97%, at least 99%, identical to SEQ ID NO: 4 which encodes SEQ ID NO: 5. In certain embodiments, the nucleic acid sequence has the nucleic acid sequence of SEQ ID NO: 4 or a sequence at least 70% to 99%, at least 75%, at least 80%, at least 85%, at least 90%, at least 95%, at least 97%, at least 99%, identical to about nt 412 to about nt 2211 of SEQ ID NO: 4 which encodes the vp2 capsid protein (about aa 138 to 736) of SEQ ID NO: 5. In certain embodiments, the nucleic acid sequence has the nucleic acid sequence of about nt 607 to about nt 2211 of SEQ ID NO: 4 or a sequence at least 70% to 99%, at least 75%, at least 80%, at least 85%, at least 90%, at least 95%, at least 97%, at least 99%, identical to nt 607 to about nt 2211 of SEQ ID NO: 4 which encodes the vp3 capsid protein (about aa 203 to 736) of SEQ ID NO: 5.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 5 provides the encoded amino acid sequence of the AAVhu68 vp1 protein. The term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

AAVrh.91 is described in WO 2020/223231, as well as in U.S. Provisional Patent Application No. 63/065,616, filed Aug. 17, 2020 and U.S. Provisional Patent Application No. 63/109,734, filed Nov. 4, 2020, each of which is incorporated by reference in its entirety herein. In certain embodiments, an AAVrh.91 capsid is characterized by one or more of the following: 1) AAVrh.91 vp1 proteins produced by expression from a nucleic acid sequence which encodes the predicted amino acid sequence of 1 to 736 of SEQ ID NO: 10, vp1 proteins produced from SEQ ID NO: 9, or vp1 proteins produced from a nucleic acid sequence at least 70% identical to SEQ ID NO: 9 which encodes the predicted amino acid sequence of 1 to 736 of SEQ ID NO: 10; 2) AAVrh.91 vp2 proteins produced by expression from a nucleic acid sequence which encodes the predicted amino acid sequence of at least about amino acids 138 to 736 of SEQ ID NO: 10, vp2 proteins produced from a sequence comprising at least nucleotides 412 to 2211 of SEQ ID NOs: 9, or vp2 proteins produced from a nucleic acid sequence at least 70% identical to at least nucleotides 412 to 2211 of SEQ ID NO: 9 which encodes the predicted amino acid sequence of at least about amino acids 138 to 736 of SEQ ID NO: 10; and/or 3) AAVhu68 vp3 proteins produced by expression from a nucleic acid sequence which encodes the predicted amino acid sequence of at least about amino acids 203 to 736 of SEQ ID NO: 10, vp3 proteins produced from a sequence comprising at least nucleotides 607 to 2211 of SEQ ID NO: 9, or vp3 proteins produced from a nucleic acid sequence at least 70% identical to at least nucleotides 607 to 2211 of SEQ ID NO: 9 which encodes the predicted amino acid sequence of at least about amino acids 203 to 736 of SEQ ID NO: 10.

In certain embodiments, an AAVrh.91 capsid is characterized by one or more of the following: 1) AAVrh.91 vp1 proteins produced by expression from a nucleic acid sequence which encodes the predicted amino acid sequence of 1 to 736 of SEQ ID NO: 10, vp1 proteins produced from SEQ ID NO: 11, or vp1 proteins produced from a nucleic acid sequence at least 70% identical to SEQ ID NO: 11 which encodes the predicted amino acid sequence of 1 to 736 of SEQ ID NO: 10; 2) AAVrh.91 vp2 proteins produced by expression from a nucleic acid sequence which encodes the predicted amino acid sequence of at least about amino acids 138 to 736 of SEQ ID NO: 10, vp2 proteins produced from a sequence comprising at least nucleotides 412 to 2211 of SEQ ID NOs: 11, or vp2 proteins produced from a nucleic acid sequence at least 70% identical to at least nucleotides 412 to 2211 of SEQ ID NO: 11 which encodes the predicted amino acid sequence of at least about amino acids 138 to 736 of SEQ ID NO: 10; and/or 3) AAVhu68 vp3 proteins produced by expression from a nucleic acid sequence which encodes the predicted amino acid sequence of at least about amino acids 203 to 736 of SEQ ID NO: 10, vp3 proteins produced from a sequence comprising at least nucleotides 607 to 2211 of SEQ ID NO: 11, or vp3 proteins produced from a nucleic acid sequence at least 70% identical to at least nucleotides 607 to 2211 of SEQ ID NO: 11 which encodes the predicted amino acid sequence of at least about amino acids 203 to 736 of SEQ ID NO: 10.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 5 (AAVhu68) may be deamidated based on the total vp1 proteins may be deamidated based on the total vp1, vp2 and vp3 proteins). Such percentages may be determined using 2D-gel, mass spectrometry techniques, or other suitable techniques.

Deamidation in the rAAV may be determined using 2D gel electrophoresis, and/or mass spectrometry (MS), and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH₂ groups). The percent deamidation of a particular peptide is determined by the mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It is understood by one of skill in the art that a number of variations on these illustrative methods can be used. For example, suitable mass spectrometers may include, e.g., a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations. Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternative one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

Nucleic acid sequences encoding the capsid of the clade F adeno-associated virus termed AAVhu68 are utilized in the production of the AAVhu68 capsid and recombinant AAV (rAAV) carrying a vector genome. The rAAVhu68.FXN described herein are well suited for delivery of the vector genome comprising the FXN gene to cardiac cells and/or cells within the central nervous system (CNS) (e.g., brain, cerebellum). In certain embodiments, an rAAVhu68.hFXN is used in combination with a second rAAV.hFXN vector having a different capsid, optionally delivered via the same route or via a different route. In certain embodiments, an rAAV.hFXN as described herein has a different capsid, which is suitable for delivering a vector genome to the CNS, cardiac, or another cell type. Suitable capsids include, for example, AAVcy02, AAV8, AAVrh43, AAV9, AAVrh08, AAVrh10, AAVbb01, AAVhu37, AAVrh20, AAVrh39, AAV1, AAVhu48, AAVcy05, AAVhu11, AAVhu32, AAVrh.91 and AAVpi02, among others.

As used herein, the term “vector genome” refers to a nucleic acid molecule which is packaged in a viral capsid, for example, an AAV capsid, and is capable of being delivered to a host cell or a cell in a patient. In certain embodiments, the vector genome is an expression cassette having inverted terminal repeat (ITR) sequences necessary for packaging the vector genome into the AAV capsid at the extreme 5′ and 3′ end and containing therebetween a FXN gene as described herein operably linked to sequences which direct expression thereof.

As used herein, an “AAV9 capsid” is a self-assembled AAV capsid composed of multiple AAV9 vp proteins. The AAV9 vp proteins are typically expressed as alternative splice variants encoded by a nucleic acid sequence of SEQ ID NO: 6 or a sequence at least 70%, at least 75%, at least 80%, at least 85%, at least 90%, at least 95%, at least 97%, at least 99% thereto, which encodes the vp1 amino acid sequence of GenBank accession: AAS99264. In certain embodiments, “AAV9 capsid” includes an AAV having an amino acid sequence which is 99% identical to AAS99264 or 99% identical to SEQ ID NO: 7. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809.

Methods of generating the capsid, coding sequences therefore, and methods for production of rAAV have been described. See, e.g., Gao, et al, Proc. Natl. Acad. Sci. U.S.A. 100 (10), 6081-6086 (2003) and US 2013/0045186A1.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

rAAVs have been previously described as suitable vehicles for gene delivery. Typically, an exogenous expression cassette comprising the transgene (for example, the FXN gene) for delivery by the rAAV replaces the functional rep genes and the cap gene from the native AAV source, resulting in a replication-incompetent vector. These rep and cap functions are provided in trans during the vector production system but absent in the final rAAV.

As indicated above, a rAAV is provided which has an AAV capsid and a vector genome which comprises, at a minimum, AAV inverted terminal repeats (ITRs) required to package the vector genome into the capsid, a FXN gene and regulatory sequences which direct expression of the FXN gene. In certain embodiments, the AAV capsid is from AAVhu68. The examples herein utilize a single-stranded AAV vector genome, but in certain embodiments, a rAAV be utilized in the invention which contains a self-complementary (sc) AAV vector genome.

The regulatory control elements necessary are operably linked to the gene (e.g., FXN) in a manner which permits its transcription, translation and/or expression in a cell which takes up the rAAV. As used herein, “operably linked” sequences include both expression control sequences that are contiguous with the gene of interest and expression control sequences that act in trans or at a distance to control the gene of interest. Such regulatory sequences typically include, e.g., one or more of a promoter, an enhancer, an intron, a polyA, and a self-cleaving linker (e.g., furin, furin-F2A, an IRES). The examples below utilize the CB7 promoter (e.g., nt 198-579 of SEQ ID NO: 8 (CMV IE promoter) through CB promoter (nt 582-863 of SEQ ID NO: 8)) for expression of the FXN gene. However, in certain embodiments, other promoters, or an additional promoter, may be selected.

In certain embodiments, in addition to the FXN gene, a non-AAV sequence encoding another one or more of gene products may be included in the vector genome. Such gene products may be, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The AAV vector genome typically includes cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external A elements is deleted. The shortened ITR is reverted back to the wild type length of 145 base pairs during vector DNA amplification using the internal A element as a template. In other embodiments, the full-length AAV 5′ and 3′ ITRs are used. See, SEQ ID NO: 8. See, also, SEQ ID NO: 12. However, ITRs from other AAV sources may be selected. Where the source of the ITRs is from AAV2 and the AAV capsid is from another AAV source, the resulting rAAV may be termed pseudotyped. However, other configurations of these elements may be suitable.

In certain embodiments, an additional or alternative promoter sequence may be included as part of the expression control sequences (regulatory sequences), e.g., located between the selected 5′ ITR sequence and the coding sequence. Constitutive promoters, regulatable promoters (see, e.g., WO 2011/126808 and WO 2013/04943), tissue specific promoters (for example, a neuron specific promoter or a glial cell specific promoter, or a CNS specific promoter), or a promoter responsive to physiologic cues may be utilized in the rAAVs described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. Other suitable promoter may include a CB7 promoter. In addition to a promoter, a vector genome may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the regulatory sequences comprise one or more expression enhancers. In one embodiment, the regulatory sequences contain two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16:605-619). In certain embodiments, no WPRE sequence is present.

In certain embodiments, vector genomes are constructed which comprise a 5′ AAV ITR-promoter-optional enhancer-optional intron-FXN gene-polyA-3′ ITR. In certain embodiments, the ITRs are from AAV2. In certain embodiments, the vector genome comprises SEQ ID NO: 8 or SEQ ID NO: 12. In certain embodiments, the vector genome comprises a 5′ ITR, nucleotides 198 to 2737 of SEQ ID NO: 8 and a 3′ ITR. In the embodiments, the vector genome comprises a 5′ ITR, nucleotides 198 to 2736 of SEQ ID NO: 12, and a 3′ ITR. In certain embodiments, more than one promoter is present. In certain embodiments, the enhancer is present in the vector genome. In certain embodiments, more than one enhancer is present. In certain embodiments, an intron is present in the vector genome. In certain embodiments, the enhancer and intron are present. In certain embodiments, the polyA is a rabbit beta-globin (RBG) poly A. In certain embodiments, the vector genome comprises a 5′ AAV ITR-CB7 promoter-FXN gene-RBG poly A-3′ ITR. In certain embodiments, the FXN gene includes SEQ ID NO: 3. In certain embodiments, the vector genome has the sequence of SEQ ID NO: 8 or a sequence at least about 85%, 90%, 91%, 92%, 93%, 94%, 95%, 96%, 97%, 98%, or 99%, to about 99.9% identical thereto. In certain embodiments, the vector genome has the sequence of SEQ ID NO: 12 or a sequence at least about 85%, 90%, 91%, 92%, 93%, 94%, 95%, 96%, 97%, 98%, or 99%, to about 99.9% identical thereto.

In certain embodiments, the vector genome further comprises a dorsal root ganglion (DRG)-specific miRNA target sequence, which allows for modulation of frataxin expression wherein expression of frataxin is repressed in DRG neurons. Such modulation of FXN transgene expression allows for decreased toxicity, thereby improving safety. See, e.g., PCT/US19/67872, filed Dec. 20, 2019 and now published as WO 2020/132455. See, also, U.S. Provisional Patent Application No. 63/023,593, filed May 12, 2020; U.S. Provisional Patent Application No. 63/038,488, filed Jun. 12, 2020; U.S. Provisional Patent Application No. 63/043,562, filed Jun. 24, 2020; and U.S. Provisional Patent Application No. 63/079,299, filed Jun. 24, 2020, which are incorporated herein by reference.

In certain embodiments, provided herein are vector genomes comprising at least one copy of DRG-specific miRNA target sequence operably linked to a FXN transgene to repress expression of the transgene in DRG and/or reduce or eliminate DRG toxicity and/or axonopathy. In certain embodiments, the vector genome comprises multiple DRG-specific miRNA target sequences, such that the number of miRNA target sequences is sufficient to reduce or minimize transgene expression in DRG to reduce and/or eliminate DRG toxicity and/or axonopathy. In some embodiments, the vector genome comprises at least two, or at least three tandem repeats of dorsal root ganglion (DRG)-specific miRNA target sequences, optionally separated by a spacer. In some embodiments, the DRG-specific miRNA target sequence/s are located at 5′ end of FXN transgene. In some embodiments, the DRG-specific miRNA target sequence/s are located at 3′ end of FXN transgene. In certain embodiments, the vector genome comprises a 5′ AAV ITR-CB7 promoter-FXN gene-one, two, or three DRG-specific miRNA targeting sequence/s-RBG poly A-3′ ITR. Such vector genome may be delivered via any suitable carrier system, viral vector or non-viral vector, via any route, but is particularly useful for intrathecal and intraparenchymal administration.

II. rAAV Production

Vector genomes for use in producing an AAV viral vector (e.g., a recombinant (r) AAV) can be carried on any suitable vector, e.g., a plasmid, which is delivered to a packaging host cell. Plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99:119-145; Buning et al., 2008, Recent developments in adeno-associated virus vector technology, J. Gene Med. 10:717-733; and the references cited below, each of which is incorporated herein by reference in its entirety. For packaging a gene into virions, the ITRs are the only AAV components required in cis in the same construct as the nucleic acid molecule containing the gene. The cap and rep genes can be supplied in trans.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product (for example, β-gal). These empty capsids are non-functional to transfer the gene of interest to a host cell. In certain embodiment, the rAAV.FXN or the composition as described herein may be at least about 80%, 85%, 90%, 95%, 96%, 97%, 98%, 99% or 99.9% free from an AAV intermediate, i.e., containing less than 20%, 15%, 10%, 5%, 4%, 3%, 2%, 1%, or 0.1% AAV intermediates.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV (such as rAAVhu68) is provided. Such a cell culture contains a nucleic acid which expresses the AAV capsid protein in the host cell; a nucleic acid molecule suitable for packaging into the AAV capsid, e.g., a vector genome which contains AAV ITRs and a FXN gene operably linked to regulatory sequences which direct expression of the gene in a cell (for example, a cell in a patient in need); and sufficient AAV rep functions and adenovirus helper functions to permit packaging of the vector genome into the recombinant AAV capsid. In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAVhu68 capsid.

Optionally the rep functions are provided by an AAV other than the capsid source AAV. In certain embodiments, at least parts of the rep functions are from AAVhu68. In another embodiment, the rep protein is a heterologous rep protein other than AAVhu68 rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a host cell.

In one embodiment, vectors are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is a rAAV and the plasmids generated are an AAV cis-plasmid encoding the AAV vector genome comprising the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the rAAV harvest, diafiltration of the rAAV harvest, microfluidization of the rAAV harvest, nuclease digestion of the rAAV harvest, filtration of microfluidized intermediate, crude purification by chromatography, crude purification by ultracentrifugation, buffer exchange by tangential flow filtration, and/or formulation and filtration to prepare bulk rAAV. A two-step affinity chromatography purification at high salt concentration followed anion exchange resin chromatography are used to purify the rAAV drug product and to remove empty capsids. These methods are described in more detail in WO 2017/160360, International Patent Application No. PCT/US2016/065970, filed Dec. 9, 2016 and its priority documents, U.S. Patent Application Nos. 62/322,071, filed Apr. 13, 2016 and 62/226,357, filed Dec. 11, 2015 and entitled “Scalable Purification Method for AAV9”, which is incorporated by reference herein.

To calculate empty and full particle content, VP3 band volumes for a selected sample (e.g., in examples herein an iodixanol gradient-purified preparation where # of genome copies (GC)=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. In certain embodiments, the AAV viral capsid purity is greater than or equal to about 90% virion protein as measured with SDS-PAGE.

Generally, methods for assaying for empty capsids and rAAV particles with packaged vector genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the rAAV is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

In brief, the method for separating rAAV particles having packaged genomic sequences from genome-deficient rAAV intermediates involves subjecting a suspension comprising rAAV viral particles and rAAV capsid intermediates to fast performance liquid chromatography, wherein the rAAV viral particles and rAAV intermediates are bound to a strong anion exchange resin equilibrated at a pH of about 10.2, and subjected to a salt gradient while monitoring eluate for ultraviolet absorbance at about 260 nanometers (nm) and about 280 nm. Although less optimal for rAAVhu68, the pH may be in the range of about 10.0 to 10.4. In this method, the AAVhu68 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/hu68 serotype. Under these ionic conditions, a significant percentage of residual cellular DNA and proteins flow through the column, while AAV particles are efficiently captured.

The rAAV.FXN (for example, rAAVhu68.FXN) is suspended in a suitable physiologically compatible composition (e.g., a buffered saline). This composition may be frozen for storage, later thawed and optionally diluted with a suitable diluent. Alternatively, the rAAV.FXN may be prepared as a composition which is suitable for delivery to a patient without proceeding through the freezing and thawing steps.

Also provided herein is a production vector (such as a plasmid) or a host cell for producing the vector genome and/or the rAAV.FXN as described herein. As used herein, a production vector carries a vector genome to a host cell for generating and/or packaging a gene therapy vector as described herein. In certain embodiments, a plasmid with an expression cassette having a FXN gene with the sequence of SEQ ID NO: 3 or a sequence 95% identical thereto that encodes human frataxin is provided. In further embodiments, the plasmid has a FXN gene that encodes a human frataxin protein having a sequence of SEQ ID NO: 2, or a sequence at least 95% identical thereto. In certain embodiments, the plasmid includes a vector genome having at least a 5′ AAV ITR, promoter, FXN gene, polyA, and a 5′ AAV ITR. In certain embodiments, the plasmid includes SEQ ID NO: 8 or 12, or a sequence at least 95% identical to SEQ ID NO: 8 or 12. In another embodiment, a host cell containing a plasmid as described herein is provided.

### III. Pharmaceutical Compositions and Methods of Treatment

Provided herein are compositions containing a rAAV and an optional carrier, excipient and/or preservative.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In particular, the compositions provided are for the treatment of FRDA. In one embodiment, the composition is suitable for administration to a patient having FRDA or a patient who is 18 months of age or younger. In one embodiment, the composition is suitable for administration to a patient having FRDA which is 16 years old or older, and wherein the onset of FRDA was at 14 years old or younger. In one embodiment, the composition is suitable for administration to a patient in need thereof to ameliorate one or more symptoms of FA, or ameliorate one or more neurological symptoms of FRDA, or ameliorate one or more cardiac symptoms of FRDA. In some embodiments, the composition is for use in the manufacture of a medicament for the treatment of FRDA. In some embodiments, the composition is for use in the manufacture of a medicament for treatment of FRDA in patients of 16 years old or older, and wherein onset of FRDA was at 14 years old or younger. In certain embodiments, the patient is 10 years of age or younger to 25 years of age or older. In certain embodiments, the patient receiving the rAAV.FXN is 10 years to 40 years of age. In certain embodiments, the patient receiving the rAAV.FXN is from 10 years to 40 years of age, from 10 years to 15 years of age, or from 15 years to 40 years of age.

In certain embodiments, the gene therapy vector provided herein is useful for treatment of neurological conditions associated with deficiencies in levels of functional frataxin in a subject. In certain embodiments, the gene therapy vector or the composition provided herein is useful for amelioration of cardiac symptoms associated with FRDA. In certain embodiments, the gene therapy vector or the composition provided herein is useful for amelioration of diabetes symptoms associated with FRDA. In certain embodiments, amelioration of the following symptoms associated with FRDA are observed following treatment. Such improvement may include, e.g., improvement in cardiac symptoms (e.g., permitting reduction or elimination of anti-arrhythmic agents and/or anti-cardiac failure medication). In certain embodiments, treatment of the subject includes dietary modification, oral hypoglycemic therapeutics, and/or insulin for controlling diabetes mellitus. In certain embodiments, vision and hearing problems in the subject may be alleviated with either corrective devices and/or drugs. In certain embodiments, the subject's intelligence remains unaffected. In certain embodiments, psychological counseling may be helpful to relieve emotional strain that affects patients and their families. In certain embodiments, speech therapy is included to help the subject maximize verbal communication skills.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

In certain embodiments, provided herein is a composition comprising a rAAV.FXN as described herein and a pharmaceutically acceptable carrier. The phrase “pharmaceutically-acceptable” refers to molecular entities and compositions that do not produce an allergic or similar untoward reaction when administered to a host.

In certain embodiments, provided herein is a composition comprising a rAAV.FXN as described herein and a delivery vehicle. Delivery vehicles such as liposomes, nanocapsules, microparticles, microspheres, lipid particles, vesicles, and the like, may be used for the introduction of the compositions of the present invention into suitable host cells. In particular, the rAAV delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject/patient, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

The rAAV.FXN is administered in sufficient amounts to transduce cells of the subject and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., brain, CSF, heart), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

Dosages of the rAAV.FXN depend primarily on factors such as the condition being treated, the age, weight and health of the patient, and can thus vary among patients. For example, a therapeutically effective human dosage of the rAAV.FXN is generally in the range of from about 25 to about 1000 microliters to about 100 mL of solution containing concentrations of from about 1×10⁹ to 1×10¹⁶ vector genome copies. In certain embodiments, a volume of about 1 mL to about 15 mL, or about 2.5 mL to about 10 mL, or about 5 mL suspension is delivered. In certain embodiments, a volume of about 1, about 2, about 3, about 4, about 5, about 6, about 7, about 8, about 9, about 10, about 11, about 12, about 13, about 14, or about 15 mL suspension is delivered.

In some embodiments, the composition is for administration in a single dose. In some embodiments, the composition is for administration via multiple routes of delivery.

In certain embodiments, delivery via intravenous administration is contemplated with a dose ranging from about 8×10¹² genome copies (GC)/kg of rAAV.FXN to about 3×10¹⁴ GC of rAAV.FXN per kg is administered. In certain embodiments, a dose is about 1×10¹³ GC/kg to about 1×10¹⁴ GC of rAAV.FXN per patient, or about 3×10¹³ GC/kg. In certain embodiments, delivery via intravenous administration is contemplated with a dose of about 3×10¹² GC/kg to about 1×10¹⁴ GC/kg, further including doses of about 3.0×10¹³ GC/kg and about 1.0×10¹³ GC/kg.

In certain embodiments, a dose from 1×10¹⁰ GC of rAAV.FXN per g brain mass (GC/g brain mass) to 3.4×10¹¹ GC/g brain mass is administered in the volume as described herein. In certain embodiments, a dose from 3.4×10¹⁰ GC/g brain mass to 3.4×10¹¹ GC/g brain mass, or from 1.0×10¹¹ GC/g brain mass to 3.4×10¹¹ GC/g brain mass, or about 1.1×10¹¹ GC/g brain mass, or from about 1.1×10¹⁰ GC/g brain mass to about 3.3×10¹¹ GC/g brain mass is administered in the volume. In certain embodiments, a dose of about 3.0×10⁹, about 4.0×10⁹, about 5.0×10⁹, about 6.0×10⁹, about 7.0×10⁹, about 8.0×10⁹, about 9.0×10⁹, about 1.0×10¹⁰, about 1.1×10¹⁰, about 1.5×10¹⁰, about 2.0×10¹⁰, about 2.5×10¹⁰, about 3.0×10¹⁰, about 3.3×10¹⁰, about 3.5×10¹⁰, about 4.0×10¹⁰, about 4.5×10¹⁰, about 5.0×10¹⁰, about 5.5×10¹⁰, about 6.0×10¹⁰, about 6.5×10¹⁰, about 7.0×10¹⁰, about 7.5×10¹⁰, about 8.0×10¹⁰, about 8.5×10¹⁰, about 9.0×10¹⁰, about 9.5×10¹⁰, about 1.0×10¹¹, about 1.1×10¹¹, about 1.5×10¹¹, about 2.0×10¹¹, about 2.5×10¹¹, about 3.0×10¹¹, about 3.3×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹ GC per gram brain mass is administered in the volume.

The replication-defective virus compositions can be formulated in dosage units to contain an amount of replication-defective virus (for example, rAAV.FXN, rAAVhu68.FXN, or rAAVhu68.CB7.FXN) that is in the range of about 1.0×10⁹ GC to about 1.0×10¹⁶ GC (to treat an subject) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is from about 700 to 1000 μL.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass.

In one embodiment, the viral constructs may be delivered in doses of from at least about least 1×10° GC to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the rAAV.FXN is employed.

In certain embodiments, the rAAV or composition may be delivered via intraparenchymal (dentate nucleus) (IDN) at a dose of about 1×10¹¹ to about 3×10¹³, or about 1 to 2×10¹³, or about 1.7×10¹³ GC in 200 μL (i.e., unilateral administration). In some embodiments, the rAAV or a composition may be delivered via IDN at a dose of about 1×10¹¹ to about 3×10¹³, or about 8×10¹² GC in 100 μL (i.e., bilateral administration). In some embodiments, the rAAV or composition may be delivered via IDN at a dose of about 3×10¹² GC.

In certain embodiments, the composition is administered in each dentate nucleus injected at a rate of 0.5 μL/min initially, and then at an increased rate of up to 5 μL/min, 10 μL/min, 15 μL/min, or 20 μL/min based or idn refers on clinician discretion during the procedure. Such procedure may take approximately 5-6 hours and the subjects are anesthetized for the duration of the procedure.

The above-described rAAV.FXN may be delivered to a subject according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.0 to 7.5, or pH 6.2 to 7.7, or pH 6.5 to 7.5, pH 7.0 to 7.7, or pH 7.2 to 7.8, or about 7.0. In certain embodiments, the formulation is adjusted to a pH of about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8. In certain embodiments, a pH of about 7.28 to about 7.32, about 6.0 to about 7.5, about 6.2 to about 7.7, about 7.5 to about 7.8, about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8 may be desired for intrathecal delivery; whereas for intravenous delivery, a pH of about 6.8 to about 7.2 may be desired. However, other pHs within the broadest ranges and these subranges may be selected for other route of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate·7H₂O), potassium chloride, calcium chloride (e.g., calcium chloride·2H₂O), dibasic sodium phosphate, and mixtures thereof, in water. Suitably, for intrathecal delivery, the osmolarity is within a range compatible with cerebrospinal fluid (e.g., about 275 milliosmoles/liter (mOsm/L) to about 290 mOsm/L); see, e.g., emedicine.medscape.com/-article/2093316-overview. Optionally, for intrathecal delivery, a commercially available diluent may be used as a suspending agent, or in combination with another suspending agent and other optional excipients. See, e.g., Elliotts B® solution [Lukare Medical]. In certain embodiments, the intrathecal final formulation buffer (ITFFB) formulation buffer comprises an artificial cerebrospinal fluid comprising buffered saline and one or more of sodium, calcium, magnesium, potassium, or mixtures thereof; and a surfactant. In certain embodiments, the surfactant comprises about 0.0005% to about 0.001% of the suspension. In a further embodiment, the percentage (%) is calculated based on weight (w) ratio (i.e., w/w). In certain embodiments, the composition containing the rAAVhu68.FXN (e.g., the ITFFB formulation) is at a pH in the range of 6.0 to 7.5, or 6.2 to 7.7, or 6.8 to 8, or 7.2 to 7.8, or 7.5 to 8. In certain embodiments, the final formulation is at a pH of about 7, or 7 to 7.4, or 7.2. In certain embodiments, for intrathecal delivery, a pH above 7.5 may be desired, e.g., 7.5 to 8, or 7.8. In certain embodiments, a pH of about 7 is desired for intrathecal delivery as well as other delivery routes.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. In certain embodiment, the aqueous solution may have a pH of 7.2. In certain embodiment, the aqueous solution may have a pH of about 7.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% poloxamer (e.g., Kolliphor®) 188. In certain embodiments, the formulation has a pH of about 7.2. In certain embodiments, the formulation has a pH of about 7. See, e.g., harvardapparatus.com/harvard-apparatus-perfusion-fluid.html. In certain embodiments, Harvard's buffer is preferred due to better pH stability observed with Harvard's buffer. The below provides a comparison of Harvard's buffer and Elliot's B buffer.

TABLE 1
Cerebrospinal Fluid (CSF) Compositions.

| Component | Units | CSF | Elliot's B | Harvard's |
|---|---|---|---|---|
| Na⁺ | mEq/L | 117-137 | 149 | 150 |
| K⁺ | mEq/L | 2.3-4.6 | 4.0 | 3.0 |
| Mg⁺ | mEq/L | 2.2 | 2.4 | 0.8 |
| Ca²⁺ | mEq/L | 2.2 | 2.7 | 1.4 |
| Cl⁻ | mEq/L | 113-127 | 132 | 155 |
| HCO₃⁻ | mEq/L | 22.9 | 22.6 | 0 |
| Phos | mg/dL | 1.2-2.1 | 1.5 | 1.0 |
| Glucose | mg/dL | 45-80 | 80 | — |
| Pluronic | % | — | 0.001% | 0.001% |
| | | | (added) | (added) |
| Osmolarity | mOsm/L | 295 | 288 | 290 |
| pH | | 7.31 | 6.0-7.5* | 7.2 |
| | | | Drift to 9+ | (titrated to) |
| | | | (8.2+ w/o titratn) | |

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. In one embodiment, the composition is formulated for administration via an intra-cisterna magna injection (ICM). In one embodiment, the composition is formulated for administration via a CT-guided sub-occipital injection into the cisterna magna.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

As used herein, the term “intraparenchymal (dentate nucleus)” or IDN refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected.

As used herein, the term “dual route(s) of delivery” refers to a route of administration for a composition comprising delivering the composition systemically (e.g., heart) and to the CNS (e.g., dentate nucleus, DRG sensory neurons, upper motor neurons).

As used herein, the term “NAb titer” refers to a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199 (3): p. 381-390, which is incorporated by reference herein.

In some embodiments, the administration of the rAAV or a composition ameliorates symptoms of FRDA, such as neurological symptoms of FRDA. In some embodiments, following treatment, the patient has one or more of increased average life span, decreased need for a feeding tube, reduction in seizure incidence and frequency, reduction in progression towards neurocognitive decline and/or improvement in neurocognitive development.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”. Typically, such an expression cassette for generating a viral vector contains the coding sequence for the gene product described herein flanked by packaging signals of the viral genome and other expression control sequences such as those described herein.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which a vector genome comprising an expression cassette containing a gene of interest (for example, FXN) is packaged in a viral capsid (e.g., AAV or bocavirus) or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

An effective amount of an rAAV or composition may be determined based on an animal model, rather than a human patient. Examples of a suitable murine or NHP model are described herein. In certain embodiments, the animal model suitable for assessing the effective amount is Fxn cardiac conditional (Fxn^{flox/null}::Ckmm-Cre), wherein the phenotype resembles the cardiac pathology of FRDA in humans. In certain embodiments, the animal model suitable for assessing the effective amount is Fxn neurological conditional (Fxn^{flox/null}::Pvalb-Cre) murine model, which exhibits exhibit similar neurodegeneration of DRG neurons and Purkinje cells accompanied by partial morphological abnormalities of mitochondria and impaired mitochondrial activity (Piguet, F et al., Rapid and complete Reversal of Sensory Ataxia by Gene Therapy in a novel Model of Freidrich Ataxia, Mol. Ther., 2018, 26 (8): P1940-1952, epub May 10, 2018). In some embodiments, the assessment in cardiac conditional Fxn cKO mice comprises the efficacy of rAAV.FXN, administered via IV, on the onset of cardiac symptoms using echocardiograms and heart succinate dehydrogenase (SDH) activity. In some embodiments, the assessment in neurological conditional Fxn KO mice evaluates the efficacy of rAAV.FXN administered, via IV, on body weight, survival, neurological and neuromuscular function endpoints. In some embodiments, the assessment comprises survival, body weight, clinical signs, cardiac function, biomarkers (e.g., growth differentiation factor 15 (GDF-15)), transgene expression (e.g., in heart, brain, DRG, spinal cord), and histopathological assessments.

A comparison of the clinical features and disease progression of these murine KO mouse models and human adult FRDA patients is presented in Table 3.

TABLE 3

| | | Biochemical | | Clinical |
|---|---|---|---|---|
| | Mutation | Abnormalities | Histopathology | Presentation |
| Fxn Cardiac | Deletion of | Reduced | Abnormal | Decrease |
| Conditional | exon 2 or 4 of | cellular | mitochondria | survival and |
| Mouse | the FXN gene | respiration | Hypertrophic | body weight, |
| | | | cardiomyopathy | progressive |
| | | | Iron accumulation | cardiomyopathy |
| | | | Cardiac fibrosis | |
| Fxn Neurologi- | Deletion of | Reduced | Abnormal | Decrease |
| cal Conditional | exon 2 or 4 of | cellular | mitochondria | survival and |
| Mouse | the FXN gene | respiration | Neurodegeneration | ataxia |
| Human FDRA | GAA repeat | Reduced | Iron accumulation | Decrease |
| Patients | expansion | cellular | Abnormal | survival, ataxia |
| | (different | respiration | mitochondria | and |
| | length) in | | Hypertrophic | cardiomyopathy |
| | intron 1 of | | cardiomyopathy | |
| | the FXN gene | | Cardiac fibrosis | |
| | | | Neurodegeneration | |

In some embodiments, rAAV.FXN is administered IV at doses of 1×10¹¹ to 1×10¹⁴ GC/kg In some embodiments, rAAV.FXN is administered IDN at a dose of about 1.5×10¹² GC bilaterally, for a total dose of 1×10¹¹ to 3×10¹² GC/kg.

In certain embodiments, the rAAV.FXN is administered by a method of dual-route administration to a patient in a need thereof, wherein a dose is administered IV, and approximately a log (10×) lower dose is administered IDN. In some embodiments, the rAAV.FXN is administered to a patient of 16 years old or older, wherein the diagnosed onset of FRDA was at 14 years old or younger at a dose as determined from the above-described scaling studies in mice and NHPs. The patient population of 16 years old or older, and wherein FRDA onset is at 14 years old or younger, presents with both the neurological and cardiac manifestations of the disease, progresses at a faster rate, and are more homogeneous in their disease presentation than late-onset patients, making them the most appropriate population for whom a stabilizing, disease-modifying therapy is most beneficial. These patients also represent a population with high unmet need. The early-onset form of FRDA has a variable age of onset occurring between 10.5-15.5 years old (Harding, 1981; Filla et al., 1990; Dürr et al., 1996; Parkinson et al., 2013). The age of disease onset is correlated to severity of disease, with younger patients generally experiencing more severe symptoms and a faster rate of disease progression (Reetz et al., 2015).

In some embodiments, a treatment regimen for FRDA comprises of rAAV.FXN administered by a method of dual-route administration to a patient in a need thereof, wherein two stocks of rAAV.FXN are used and one of the rAAV.FXN includes a vector genome having one or more DRG-specific miR target sequences (as described above). In certain embodiments, a rAAV.FXN is delivered by IV, and a rAAV.FXN having a vector genome with one or more DRG-specific miR target sequences is delivered by IDN. In another embodiment, the rAAV.FXN having a vector genome with one or more DRG-specific miR target sequences is delivered IV, and another rAAV.FXN is delivered IDN. In certain embodiments, two different stocks of rAAV.FXN are utilized, which may have miR target sequences which are the same, or which differ from each other. In certain embodiments, the rAAV.FXN have different capsids.

Optionally, an immunosuppressive co-therapy may be included in the treatment of a subject in need. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, cyclosporin, tacrolimus, sirolimus, IFN-β, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed.

Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in U.S. Provisional Patent Application No. 63/040,381, filed Jun. 17, 2020, entitled “Compositions and Methods for Treatment of Gene Therapy Patients”, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

### IV. Apparatus and Method for Delivery of a Pharmaceutical Composition

In one aspect, the rAAV or composition provided herein may be administered intrathecally via the method and/or the device provided in this section and described in WO 2018/160582, which is incorporated by reference herein. Alternatively, other devices and methods may be selected.

In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis.

On the day of treatment, the appropriate concentration of rAAV.FXN is be prepared. A syringe containing 5.6 mL of rAAV.FXN at the appropriate concentration is delivered to the procedure room. The following personnel are present for study drug administration: interventionalist performing the procedure; anesthesiologist and respiratory technician(s); nurses and physician assistants; CT (or operating room) technicians; site research coordinator. Prior to drug administration, a lumbar puncture is performed to remove a predetermined volume of CSF and then to inject iodinated contrast intrathecally (IT) to aid in visualization of relevant anatomy of the cisterna magna. Intravenous (IV) contrast may be administered prior to or during needle insertion as an alternative to the intrathecal contrast. The decision to used IV or IT contrast is at the discretion of the interventionalist. The subject is anesthetized, intubated, and positioned on the procedure table. The injection site are prepped and draped using sterile technique. A spinal needle (22-25 G) are advanced into the cisterna magna under fluoroscopic guidance. A larger introducer needle may be used to assist with needle placement. After confirmation of needle placement, the extension set are attached to the spinal needle and allowed to fill with CSF. At the discretion of the interventionalist, a syringe containing contrast material may be connected to the extension set and a small amount injected to confirm needle placement in the cisterna magna. After the needle placement is confirmed by CT guidance+/−contrast injection, a syringe containing 5.6 mL of rAAV.FXN is connected to the extension set. The syringe contents are slowly injected over 1-2 minutes, delivering a volume of 5.0 mL. The needle is slowly removed from the subject.

In one aspect, the rAAV or composition provided herein may be administered via intraparenchymal (dentate nucleus) (IDN) route by a method and/or the device using ClearPoint® Neuro Navigation System and ventricular cannula. Alternatively, other devices and methods may be selected. In some embodiments, the rAAV or compositions are administered via IDN to address neurological manifestation of FRDA.

In certain embodiments, the methods comprises using the ClearPoint® injection system wherein the system consists of a monitor to visualize the brain and injection procedure in real time, a head fixation frame that is secured to the skull, and an MRI-compatible SmartFrame® (MRI Interventions Inc., Memphis, TN) trajectory device that enables MRI-guided alignment during the procedure. This system allows for the direct injection to be combined with real-time visualization of the injection tract by MRI. To enable visualization of rAAV or composition distribution, the injection material containing the vector is mixed with gadolinium, which is contrast agent (final concentration of 1-2 mM gadolinium). During the direct injection procedure, the injection cannula is placed through the ClearPoint® frame to the correct position on the skull and the frame maintains the correct trajectory. The final position of the injection cannula is confirmed using real-time MRI images, and then the rAAV or composition is injected into the parenchyma of the deep cerebellar nuclei using convection-enhanced delivery. Each subject receives administration of the rAAV or composition plus gadolinium in each dentate nucleus injected at a rate of 0.5 μL/min initially, and then at an increased rate of up to 5 μL/min based on clinician discretion during the procedure. The procedure takes approximately 5-6 hours and subjects are anesthetized for the duration of the procedure.

In certain embodiments, the rAAV or composition is administered intravenously (IV). In some embodiments, IV administration is by IV infusion into peripheral vein. In some embodiments, the IV infusion rate and/or time is determined from nonclinical NHP studies, as described herein. In some embodiments, the IV infusion is over no less than a 20-minute interval using a syringe infusion pump via an IV administration set. In some embodiments, the IV infusion is 20-minites to 1-hour long. In some embodiments, the IV infusion is 1-hour or longer, as per discretion of investigator, wherein a lower infusion rate may be necessary. The IV infusion occurs no longer than 24 hours prior to the IDN procedure occurring the following day. In some embodiments, the IV administration allows for observations of acute hypersensitivity to the rAAV or composition. In some embodiments, the rAAV or composition is administered by IV to address cardiac manifestation of FRDA.

In certain embodiments, the rAAV or composition provided herein may be administered via a method of dual-route administration comprising intravenous (IV) and intraparenchymal (dentate gyrus) (IDN), as two sequential doses within 24 hours of one another. The dual route of administration target peripheral organs, i.e., cardiac myocytes (i.e., IV) and central organs, cerebellum and sensory DRG neurons (i.e., IDN). In some embodiments, the IDN administration is unilateral. In some embodiments, the IDN administration is bilateral. In some embodiments, the rAAV or composition is administered via unilateral and/or bilateral MRI guided direct injection into the deep cerebellar nuclei (DCN) via convection-enhanced delivery (CED). In some embodiments, the rAAV is administered at a dose of 3.0×10¹³ GC/kg via IV and at a dose of 1.5×10¹² GC in 50 μL via IDN (bilaterally, total dose of 3.0×10¹² GC/kg). The volume of IV infusion is determined based on the dose level and the weight of the subject. In some embodiments, the rAAV or composition is administered via IV and IDN (i.e., dual-routes of administration) to address both cardiac and neurological manifestations of FRDA. In some embodiments, the rAAV or composition is delivered via dual-routes of administration, wherein the amount of vector delivered by IV to the amount of vector delivered by IDN is at the ratio of about 10 to about 1. In further embodiments, the ratio of vector delivered by IV to vector delivered by IDN comprises of ration of about 2:1 to about 8:1, or about 3:1 to about 5:1, and inclusive of the values in between.

In certain embodiments, the efficacy of dual routes of administration of a rAAV or a composition is determined through the following endpoints:
General:
   Survival
   Levels of frataxin expression in serum samples evaluated at 1-year rAAV or composition treatment
   Quality of Life (QoL) as assessed by a FRDA-specific patient reported outcome questionnaire currently being developed by the Friedreich's Ataxia Research Alliance at 2 years post-rAAV or composition treatment
Neurological Endpoints:
   mFARS assessment as compared to baseline score to measure overall disease state over time (FARS scale is an exam-based rating scale that assesses neurological function over 5 areas of disease involvement (bulbar, upper limb, lower limb, peripheral nervous system, and upright stability) (Subramony et al., 2005)).
   Fine motor skills assessment with a 9-hole peg test (9HPT) measuring if subject is able to perform in <5 minutes; or use a spoon dexterity test if subject is unable to complete the 9HPT,
   Ambulation assessment by a 25-foot walk test at 2 years post-rAAV or composition treatment
   Dysarthia assessment (e.g., via speech analysis software)
Cardiac Endpoints:
   Electrocardiogram changes
   Cardiac MRI assessment
   Absence of progression of cardiac symptoms, including ICD and heart failure hospitalization.

Additional or alternate routes of administration to the intrathecal method described herein include, for example, systemic, oral, intravenous, intraperitoneal, subcutaneous, or intramuscular administration.

In one embodiment, doses may be scaled by brain mass, which provides an approximation of the size of the CSF compartment. In a further embodiment, dose conversions are based on a brain mass of 0.4 g for an adult mouse, 90 g for a juvenile rhesus macaque, and 800 g for children 4-18 months of age. The following Table 2 provides illustrative doses for a murine MED study, NHP toxicology study, and equivalent human doses.

TABLE 2
Illustrative doses for study in mouse and NHP.

| Dose | | | |
|---|---|---|---|
| (GC/g brain | | | |
| mass) | Mouse (GC) | NHP (GC) | Human (GC) |
| 3.33 × 10¹¹ | 1.30 × 10¹¹ | 3.00 × 10¹³ | 2.70 × 10¹⁴ |
| 1.11 × 10¹¹ | 4.40 × 10¹⁰ | 1.00 × 10¹³ | 8.90 × 10¹³ |
| 3.33 × 10¹⁰ | 1.30 × 10¹⁰ | 3.00 × 10¹² | 2.70 × 10¹³ |
| 1.11 × 10¹⁰ | 4.40 × 10⁹ | — | 8.90 × 10¹² |

In certain embodiments, a rAAV.FXN is administered to a subject in a single dose. In certain embodiments, the concentration in GC is illustrated as GC per spinal tap. In certain embodiments, the concentration in CG is illustrated as GC per mL.

A co-therapy may be delivered with the rAAV.FXN compositions provided herein. Co-therapies such as described earlier in this application are incorporated herein by reference.

### EXAMPLES

The following examples are illustrative only and are not intended to limit the present invention.

| Abbreviation | Description |
|---|---|
| A | Absorbance |
| aa | Amino Acids |
| AE | Adverse Events |
| AEX | Anion Exchange |
| AmpR | Ampicillin Resistance (gene) |
| AUC | Analytical Ultracentrifugation |
| BA | Chicken β-Actin |
| BAER | Brainstem Auditory Evoked Response |
| BBB | Blood-Brain Barrier |
| BCA | Bicinchoninic Acid |
| BMT | Bone Marrow Transplant |
| bp | Base Pairs |
| BSA | Bovine Serum Albumin |
| BSE | Bovine Spongiform Encephalopathy |
| BSID-III | Bayley Scales of Infant and Toddler Development, |
| | Third Edition |
| BWCB | Bacterial Working Cell Bank |
| cap | Capsid (gene) |
| cDNA | Complementary Deoxyribonucleic Acid |
| CFR | Code of Federal Regulations |
| CFU | Colony Forming Units |
| cGMP | Current Good Manufacturing Practice |
| Cho | Choline |
| CI | Chimeric Intron |
| CMC | Chemistry Manufacturing and Controls |
| CMO | Contract Manufacturing Organization |
| CMV IE | Cytomegalovirus Immediate-Early Enhancer |
| CNS | Central Nervous System |
| CPE | Cytopathic Effects |
| CRISPR-Cas9 | Clustered Regularly Interspaced Short Palindromic |
| | Repeats/CRISPR-Associated Protein 9 |
| CRL | Charles River Laboratories |
| CSF | Cerebrospinal Fluid |
| CT | Computed Tomography |
| ddPCR | Droplet Digital Polymerase Chain Reaction |
| DLS | Dynamic Light Scattering |
| DMEM | Dulbecco's Modified Eagle Medium |
| DNA | Deoxyribonucleic Acid |
| DO | Dissolved Oxygen |
| DP | Drug Product |
| DRG | Dorsal Root Ganglia |
| DS | Drug Substance |
| DSMB | Data and Safety Monitoring Board |
| DT-MRI | Diffusion-Tensor Magnetic Resonance Imaging |
| E1A | Early Region 1A (gene) |
| ECG | Electrocardiogram |
| EDTA | Ethylenediaminetetraacetic Acid |
| ELISA | Enzyme-Linked Immunosorbent Assay |
| ELISpot | Enzyme-Linked Immunospot |
| ERT | Enzyme Replacement Therapy |
| EU | Endotoxin Units |
| F | Female |
| FBS | Fetal Bovine Serum |
| FDP | Filled Drug Product |
| FFB | Final Formulation Buffer |
| FIH | First-in-Human |
| GC | Genome Copies |
| GFP | Green Fluorescent Protein |
| GLP | Good Laboratory Practice |
| GMP | Good Manufacturing Practice |
| GvHD | Graft Versus Host Disease |
| HCDNA | Host Cell Deoxyribonucleic Acid |
| HCP | Host Cell Protein |
| HD | High Dose |
| HEK293 | Human Embryonic Kidney 293 |
| ICH | International Council for Harmonisation |
| ICV | Intracerebroventricular |
| IDN | Intraparenchymal (dentate gyrus) |
| IFN-γ | Interferon Gamma |
| IND | Investigational New Drug Application |
| IT | Intrathecally |
| ITFFB | Intrathecal Final Formulation Buffer |
| ITR | Inverted Terminal Repeat |
| IU | Infectious Unit |
| IV | Intravenous |
| KanR | Kanamycin Resistance (gene) |
| Lac | Lactate |
| LAL | Limulus Amoebocyte Lysate |
| LFTs | Liver Function Tests |
| LOD | Limit of Detection |
| LTFU | Long-Term Follow-Up |
| M | Male |
| MBR | Master Batch Record |
| MCB | Master Cell Bank |
| MD | Mid-dose |
| MED | Minimum Effective Dose |
| MRI | Magnetic Resonance Imaging |
| mRNA | Messenger Ribonucleic Acid |
| MRS | Magnetic Resonance Spectroscopy |
| MS | Mass Spectrometry |
| MTD | Maximum Tolerated Dose |
| N | Number of Subjects or Animals |
| N/A | Not Applicable |
| NAbs | Neutralizing Antibodies |
| NCV | Nerve Conduction Velocity |
| NGS | Next-Generation Sequencing |
| NHP | Non-Human Primate |
| NOAEL | No-Observed-Adverse-Effect Level |
| OL | Open-Label |
| PBS | Phosphate-Buffered Saline |
| PEI | Polyethylenimine |
| PES | Polyethersulfone |
| PI | Principal Investigator |
| POC | Proof-of-Concept |
| PolyA | Polyadenylation |
| QA | Quality Assurance |
| QC | Quality Control |
| qPCR | Quantitative Polymerase Chain Reaction |
| rAAV | Recombinant Adeno-Associated Virus |
| rcAAV | Replication-Competent Adeno-Associated Virus |
| rBG | Rabbit β-Globin |
| rDNA | Ribosomal Deoxyribonucleic Acid |
| rep | Replicase (gene) |
| RNA | Ribonucleic Acid |
| RPM | Revolutions Per Minute |
| SAE | Serious Adverse Events |
| SDS | Sodium Dodecyl Sulfate |
| SDS-PAGE | Sodium Dodecyl Sulfate Polyacrylamide Gel |
| | Electrophoresis |
| SMN | Survival Motor Neuron (gene) |
| SOP | Standard Operating Procedure |
| SRT | Safety Review Trigger |
| ssDNA | Single-Stranded Deoxyribonucleic Acid |
| TBD | To Be Determined |
| TCID₅₀ | 50% Tissue Culture Infective Dose |
| TE | Tris-EDTA |
| TFF | Tangential Flow Filtration |
| TSE | Transmissible Spongiform Encephalopathy |
| UPLC | Ultra-Performance Liquid Chromatography |
| US | United States |
| USP | United States Pharmacopeia |
| WCB | Working Cell Bank |
| WHO | World Health Organization |

### Example 1—Recombinant AAVhu68.hFXN

rAAVhu68.CB7.CI.hFXN.polyA (also rAAVhu68.hFXN) includes the coding sequence for human frataxin, regulatory element derived from the chicken β-actin (BA) promoter and human cytomegalovirus immediate-early enhancer (CMV IE), chimeric intron consisting of a chicken BA splice donor and a rabbit β-globin (rBG) splice acceptor element polyadenylation (PolyA) signal derived from the rBG gene, two inverted terminal repeat sequences (ITRs). Vectors are constructed from cis-plasmids containing a coding sequence for human FXN (SEQ ID NO: 3) expressed from the chicken beta actin promoter with a cytomegalovirus enhancer (CB7) flanked by AAV2 inverted terminal repeats. The vectors are packaged in an AAV serotype hu68 capsid (WO 2018/160582) by triple transfection of adherent HEK 293 cells and purified by iodixanol gradient centrifugation as previously described in Lock, M., et al. Rapid, Simple, and Versatile Manufacturing of Recombinant Adeno-Associated Viral Vectors at Scale. Human Gene Therapy 21, 1259-1271 (2010). More particularly, AAV.CB7.CI.hFXN is produced by triple plasmid transfection of HEK293 working cell bank (WCB) cells with the AAV cis plasmid (pENN.AAV.CB7.CI.hFXN), the AAV trans plasmid encoding the AAV2 rep and AAVhu68 cap genes (pAAV2/hu68.KanR), and the helper adenovirus plasmid (pAdΔF6.KanR). The AAV hu68 capsid proteins are provided in SEQ ID NO: 5. The CB7.CI.hFXN packaged vector genome is provided in SEQ ID NO: 12, which is 2954 bases. SEQ ID NO: 12 comprises a shortened AAV2 ITR sequence of 130 base pairs, wherein external A element is deleted compared to the wild type ITR sequence, which is 145 base pairs. The shortened ITR sequence is reverted back to the wild type length of 145 base pairs during vector DNA amplification using the internal A element as a template, therefore producing a vector genome having a predicted size of 2984 bases.

More detailed, the cis plasmid contains the following vector genome sequence elements:

Inverted Terminal Repeat (ITR): The ITRs are identical, reverse complementary sequences derived from AAV2 (130 base pairs [bp], GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.
Human Cytomegalovirus Immediate-Early Enhancer (CMV IE): This enhancer sequence obtained from human-derived CMV (382 bp, GenBank: K03104.1) increases expression of downstream transgenes;
Chicken β-Actin Promoter (BA): This ubiquitous promoter (281 bp, GenBank: X00182.1) was selected to drive transgene expression in any CNS cell type;
Chimeric Intron (CI): The hybrid intron consists of a chicken β-actin splice donor (973 bp, GenBank: X00182.1) and rabbit β-globin splice acceptor element. The intron is transcribed, but removed from the mature mRNA by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This feature in gene vectors intended for increased levels of gene expression;
Coding sequence: The engineered cDNA of the human FXN gene encodes human frataxin protein, which is located in mitochondria and plays a role in iron biosynthesis and chaperon (630 bp; 210 amino acids [aa], GenBank: NP_000135); and
Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

Alternatively, the manufacturing process for rAAvhu68.hFXN involves transient transfection of human embryonic kidney 293 (HEK293) cells with plasmid DNA. The HEK293 master cell bank (MCB) used in the production has been tested and qualified as detailed in FDA and International Council for Harmonization (ICH) guidelines. To support clinical development, a single batch or multiple batches of the bulk drug substance (BDS) is produced by polyethylenimine-(PEI-) mediated triple transfection of HEK293 cells in bioreactors. Harvested AAV material is purified sequentially by clarification, tangential flow filtration (TFF), affinity chromatography, and anion exchange chromatography in disposable, closed bioprocessing systems where possible. The rAAVhu68.hFXN which are used for both the IV and IDN administrations are formulated in Intrathecal Final Formulation Buffer (ITFFB): 1 mM sodium phosphate, pH 7.2, 150 mM NaCl, 3 mM KCl, 1.4 mM CaCl2, 0.8 mM MgCl2, 0.001% Poloxamer 188. The rAAVhu68.hFXN is manufactured from BDS batch or batches that is frozen, subsequently thawed, pooled if necessary, adjusted to the target concentration, and sterile-filtered through a 0.2 μm filter, and filled into vials.

Additionally, a controlled bioreactor platform is implemented: where small-scale bioreactor is a linearly scaled with the respect of the cell growth surface to the large-scale bioreactor. The use of the small-scale bioreactor and the large-scale bioreactor allows for scalable manufacturing with minimal process and material impact. The large-scale bioreactor and/or the small-scale bioreactor are utilized for the production of the toxicology lot(s). The large-scale bioreactor is used for the production of the good manufacturing practice (GMP) drug substance (DS) lot(s). Large-scale GMP production batch sizes are be generated with multiple batches planned and pooled, if necessary, to satisfy the needed vector amount for drug product (DP) supply. When transferring manufacturing to the CMO for clinical use, the critical quality attributes are anticipated to not be modified. Critical source materials remains the same, including the MCB and source of the Fetal Bovine Serum (FBS) although the PEI and plasmid DNA utilized for GMP manufacturing is produced in a manner that is designed to meet the requirements for current good manufacturing practice (cGMP) intermediates.

As the scale-up manufacturing process to the large-scale bioreactor is implemented and further optimized, and based on the combined manufacturing experience in the current bioreactor platform any potential impact related to changes in the process through comparability testing to ensure there is no change to identity, purity, potency, and safety of the product are addressed. The comparability testing that is conducted to compare a new lot manufactured with an updated procedure or with new material to a previous lot consists of a subset of tests included in the certificate of analysis (COA).

### Example 2—rAAVhu68.hFXN Delivery in Mouse Models of Friedreich's Ataxia Fxn cKO Mouse Studies (Cardiac Conditional Fxn Mouse Model)

A. Natural History Study in the Cardiac Conditional Fxn Knockout Mouse (Fxn cKO) (Nonclinical Study 1)

The activity of a rAAV.hFXN via intravenous administration has been evaluated in a murine model of FRDA cardiomyopathy.

The purpose of this non-GLP-compliant natural history study (Nonclinical Study 1) was to establish the natural disease progression of the Fxn cKO mouse model (Fxn^{flox/null}::Ckmm-Cre). Thirteen newborn (PND 0) mice were enrolled in the study, including Fxn cKO mice (Fxn^{flox/null}::Ckmm-Cre) displaying the disease phenotype and Fxn unaffected control littermates (Fxn^{flox/null}). Weekly body weights were recorded, and animals euthanized upon reaching a humane endpoint defined by weight loss ≥20% of maximal body weight. Survival was recorded.

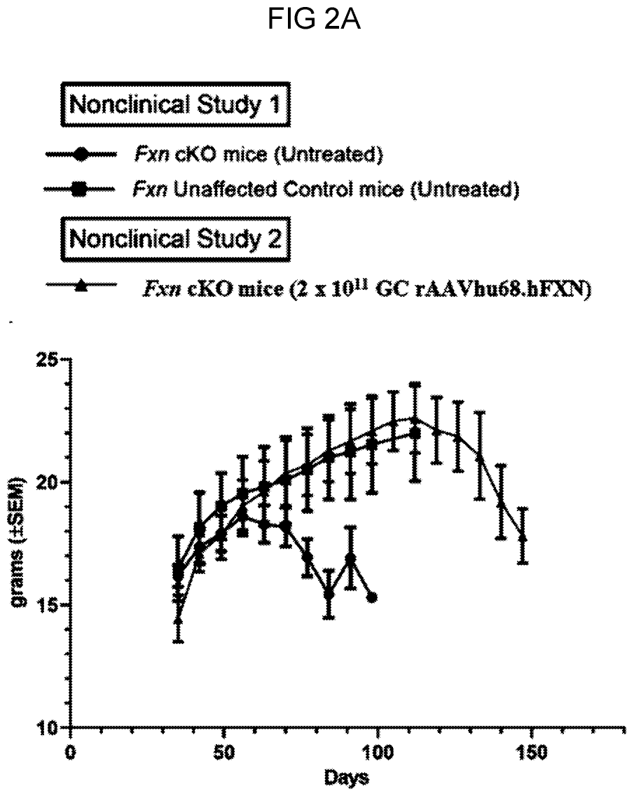

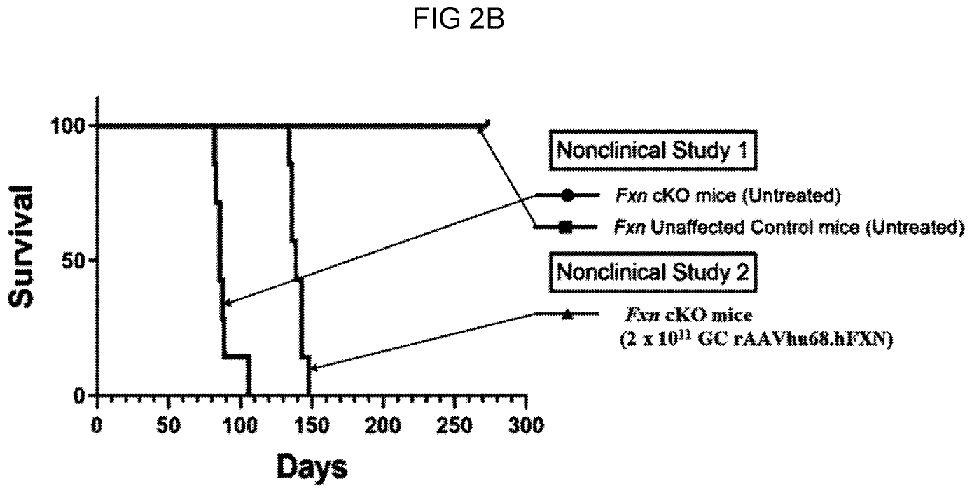

Body weight of untreated Fxn cKO mice peaked at 18.6 g by 60 days of age (8 weeks of age), after which, the mice started to lose weight until they reached a humane endpoint (FIG. 2A). This study confirmed previous reports in the Fxn cKO mice regarding initial body weight loss by 9 weeks of age (˜63 days of age) and a mean survival of 89 days of age.

### B. Study in Cardiac Conditional Fxn Knockout Mice (Nonclinical Study 2)

The objective of this non-GLP-compliant pilot POC study was to determine the survival of Fxn cKO mice (Fxn^{flox/null}::Ckmm-Cre) following IV administration of rAAVhu68.hFXN. Adult (31-34 days of age) Fxn cKO mice were administered rAAVhu68.hFXN at an IV dose of 2.0×10¹¹ GC. The dose was selected based on experience with similar AAV therapies where this dose was found to be non-toxic and lead to efficacy. The age (31-34 days of age) was selected to increase the likelihood of observing disease rescue and mirrors the intended clinical trial population. Weekly body weights were recorded. Animals were euthanized upon reaching a humane endpoint defined by weight loss ≥20% of maximal body weight, and survival was recorded. At the time of euthanasia, blood was collected for GDP-15, a measurement of cardiac stress, analysis via an ELISA assay. IV administration of rAAVhu68.hFXN, delayed body weight loss and extended survival to 140 days compared to 89 days in the untreated controls in the natural history study (Nonclinical Study 1).

Fxn cKO mice administered rAAVhu68.hFXN gained weight during the study and had comparable body weight to Fxn unaffected control mice until 120 days of age (17 weeks of age; FIG. 2A). Average weights for the Fxn cKO treated mice was 22.6 g and Fxn unaffected control mice was 21.98 g (Nonclinical Study 1). Fxn cKO mice administered rAAVhu68.hFXN began to loss weight after 120 days of age with the weight loss continuing until 150 days of age (21 weeks of age) when all mice reached humane endpoint criteria.

FIG. 2A provides the results of a pilot cardiac gene therapy study (Nonclinical Study 1 and 2) in a Fxn^{flox/null}::Ckmm-Cre FA mouse model. FIG. 2A provides a graph of body weight (grams±SEM) over time (0-20 weeks). The triangles represent Fxn^{flox/null}::Ckmm-Cre rAAVhu68.hFXN (2×10¹¹ genome copies (GC) intravenous (iv) at 5 weeks (n=7). The circles represent results in Ckmm-cre; fxn−/flox (n=7). The squares represent Ckmm-cre; fxn−/flox (n=7). At 30 days of age Fxn cKO mice were IV-administered rAAVhu68.hFXN at a dose of 2.0×10¹¹ GC (Nonclinical Study 2). Age-matched Fxn cKO mice and Fxn unaffected mice remained untreated and served as controls (Nonclinical Study 1).

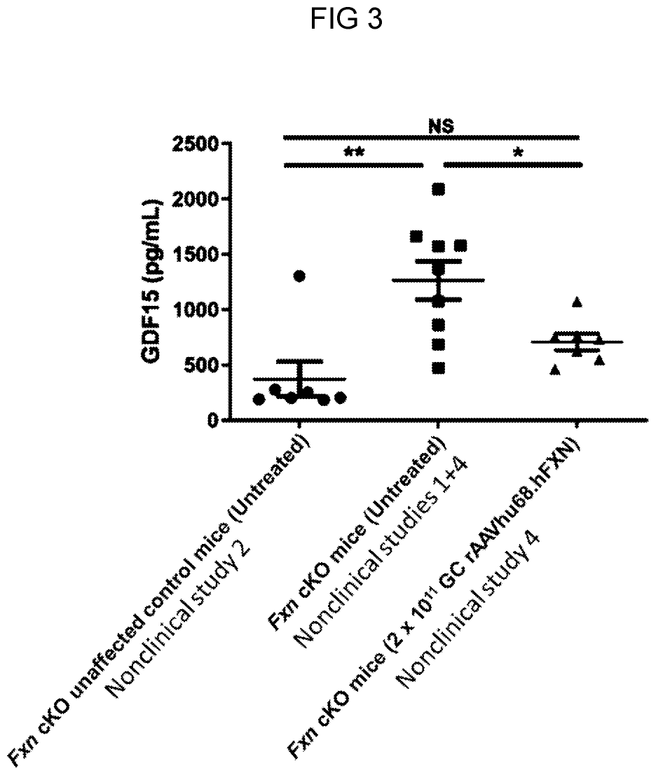

rAAVhu68.hFXN IV administration to Fxn cKO mice reduced GDF-15 serum levels indicating normalization of cardiac stress (FIG. 3).

These data demonstrated that the administration of rAAVhu68.hFXN to cFxn cKO mice resulted in delayed weight loss, improved survival, and normalization of GDF-15 levels.

### C. Survival Study in Cardiac Conditional Fxn Knockout Mice (Nonclinical Study 3)

This non-GLP-compliant study evaluated if a higher IV dose of rAAVhu68.hFXN could further extend survival in Fxn cKO (Fxn^{flox/null}::Ckmm-Cre) mice. Fxn cKO mice (26-29 days of age) were administered rAAVhu68.hFXN IV at a dose of 5.0×10¹¹ GC. The IV dose was selected based on experience with similar AAV therapies where this dose was found to be non-toxic but was efficacious. Treatment at 26-29 days of age was selected to increase the likelihood of observing disease rescue and mirrors the intended early adulthood population for the clinical trial. Animals were euthanized upon reaching a humane endpoint (defined by weight loss), and survival was recorded. IV administration of rAAVhu68.hFXN extended survival to 196 days compared to 140 days for 2.0×10¹¹ GC GTP-212 treated mice (Nonclinical Study 2).

This study demonstrated that increasing the of rAAVhu68.hFXN could further increase survival in Fxn cKO mice.

### D. Pharmacology Study in Cardiac Conditional Fxn Knockout Mice (Nonclinical Study 4)

The purpose of this non-GLP-compliant study was to evaluate the efficacy of rAAVhu68.hFXN administered IV in the Fxn cKO (Fxn^{flox/null}::Ckmm-Cre) mouse model. Adult (30 days of age) Fxn cKO mice were administered rAAVhu68.hFXN at a dose of 2.0×10¹¹ GC. Age-matched control animals included, untreated Fxn cKO and untreated Fxn unaffected control (Fxn^{flox/null}) mice. At the time of euthanasia (80 days of age), the heart was harvested for assessment of iron accumulation and blood was collected for GDF-15, a measurement of cardiac stress, analysis using an ELISA assay.

Administration of rAAVhu68.hFXN led to an increase in survival of Fxn cKO mice compared to untreated Fxn cKO mice. Untreated FXN cKO mice had an average life span of 89 days, FXN cKO mice treated with 2e13 GC hu68.hFXN had an average life span of 140 days. The 2e11 GC treated mouse that survived the longest reached 148 days of age. (FIG. 2B). Fxn unaffected control mice survived to 273 days of age when the study was terminated.

rAAVhu68.hFXN administration to Fxn cKO mice reduced GDF-15 circulating serum levels to untreated Fxn unaffected control mice levels (FIG. 3). The reduction indicates normalization of cardiac stress. Circulating GDF-15 levels in the serum were increased in untreated Fxn cKO mice compared to untreated Fxn unaffected control mice.

rAAVhu68.hFXN administered IV at a dose of 2.0×10¹¹ GC to Fxn cKO mice resulted in delayed body weight loss through 120 days of age (17 weeks of age), increased survival to 148 days of age, and normalized serum levels of GDF-15.

### E. Study in Cardiac Conditional Fxn Knockout Mice

The objectives of this non-GLP-compliant study are to: 1) establish when cardiac symptoms begin to manifest in the Fxn cKO mice (Fxn^{flox/null}::Ckmm-Cre); and 2) to determine the efficacy of IV administration of rAAVhu68.hFXN to correct cardiac pathology without leading to cardiomyopathy. For the first study objective, adult Fxn cKO mice (Fxn^{flox/null}::Ckmm-Cre) (Group 5) and Fxn unaffected Cre mice (Fxn^{+/flox}::Ckmm-Cre; Group 6) are enrolled in the study when they are 21 days of age (Table 4). Echocardiogram is performed when mice are 28 days of age to assess hypertrophic cardiomyopathy. Hypertrophic cardiomyopathy such as cardiac output, left ventricle mass, end-systolic dimension, and shortening fraction has been previously reported (Belbellaa et al., 2019). After echocardiograms at 28 days of age mice are necropsied and hearts harvested for histopathological assessment of cardiac pathology, such as fibrosis, and a disease-relevant biomarker, SDH (mitochondrial respiratory complex II) activity.

For the second study objective, adult Fxn cKO mice that are 21 or 28 days of age are IV administered rAAVhu68.hFXN at a dose 3×10¹³ GC/kg or vehicle (Table 4). Age-matched Fxn cKO and Fxn unaffected Cre mice are administered vehicle at 21 days of age as controls. Body weights and blood are collected during the study to evaluate circulating levels of GDF-15, a cardiac stress marker. At 70 days of age all mice have echocardiogram assessments to assess hypertrophic cardiomyopathy and then necropsied and hearts harvested for histopathological assessment of cardiac pathology and changes in SDH activity. Comparison of these parameters are used to assess the efficacy of GTP-212 treatment on cardiac pathology of different ages where there is the possibility of overexpression of frataxin.

TABLE 4
Group Designations.

| Group | Study Objective 2 | | | | Study Objective 1 | |
|---|---|---|---|---|---|---|
| Number | 1 | 2 | 3 | 4 | 5 | 6 |
| Animals | 21 (±2) | 21 (±2) | 21 (±2) | 21 (±2) | 21 (±2) | 21 (±2) |
| age at | days | days | days | days | days | days |
| enrollment | | | | | | |
| Genotype | Fxn cKO | Fxn cKO | Fxn | Fxn | Fxn | Fxn |
| | | | cKO | unaffected | cKO | unaffected |
| | | | | controls | | controls |
| Treatment | rAAVhu68.hFXN | rAAVhu68.hFXN | Vehicle | Vehicle | Untreated | Untreated |
| | | | (PBS) | (PBS) | | |
| ROA | IV | IV | IV | IV | NA | NA |
| Administration | 21 (±2) | 28 (±2) | 21 (±2) | 21 (±2) | NA | NA |
| Day | days old | days old | days old | days old | | |
| Dose | 3 × 10¹³ | 3 × 10¹³ | NA | NA | NA | NA |
| (GC/kg) | | | | | | |
| Necropsy | 70 (±5) | 70 (±5) | 70 (±5) | 70 (±5) | 28 (±4) | 28 (±4) |
| Day | days of age | days of | days of | days of | days of | days of |
| | | age | age | age | age | age |
| N | 8 | 8 | 8 | 8 | 8 | 8 |

Abbreviations:
Fxn, frataxin (gene, mouse);
GC, genome copies;
Fxn cKO, cardiac conditional knockout affected mice (Fxn^{flox/null}::Ckmm-Cre);
Fxn unaffected controls (Fxn^{flox/null});
GC, genome copies;
IV, intravenous,
N, number of animals;
NA, not applicable;
PBS, phosphate buffered saline;
ROA, route of administration.

F. Survival Study in Neurological Conditional Fxn Knockout Mice (Fxn ncKO) (Nonclinical Study 6)

The purpose of this non-GLP-compliant study is to determine the efficacy of IV administration rAAVhu68.hFXN on survival and ataxic behavior in the Fxn ncKO mouse model. Fxn ncKO mice (Fxn^{flox/null}:Pvalb-Cre) 31 days of age are administered rAAVhu68.hFXN IV at a dose of 3.0×10¹³ GC/kg GC or vehicle (PBS). Age-matched wild type mice are also be IV administered vehicle (PBS) as a control. The dose of rAAVhu68.hFXN was selected based on robust heart and DRG transduction observed in Nonclinical Study 7 (data not shown). Body weights are assessed weekly throughout the study. Neurological assessment (Neuroscore) is performed at baseline and weekly thereafter (Table 5). Briefly, mice are suspended by the tail to assess collapse of leg extension towards the lateral midline. Mice are then placed in a cage where the other ataxic phenotype are assessed.

Neuromotor function (rotarod) is performed at 56 days of age and every 4 weeks. Briefly, mice are habituated to the RotaRod then testing trials are performed to measure how long each mouse can remain on the rotating rod while it is accelerating. For each animal, the testing trial is considered terminated when the mouse falls off the rod, completes two passive revolutions, or 300 seconds elapses. The fall latency (defined as the time between the initiation of rod acceleration and trial termination) is then recorded. A total of three sequential test replicates are performed for the mice in each trial, with a 2 minute pause between runs to allow the animals to rest in the collecting box.

All assessments are performed until the mice reach the humane euthanasia endpoint which is defined by weight loss ≥20% of maximal body weight or a Neuroscore of 4.

TABLE 5
Neuroscore Assessment

| Observation | Score |
|---|---|
| Mouse walking/behaving normally. Full extension of | 0 |
| hind legs away from lateral midline when mouse is | |
| suspended by its tail, and mouse can hold this for | |
| 2 seconds, suspended 2-3 times. | |
| Collapse or partial collapse of leg extension towards | 1 |
| lateral midline (weakness) or trembling of hind | |
| legs during suspension. Possible minor head bobble | |
| behavior. | |
| Toes curl under at least twice during walking of 12 | 2 |
| inches, or any part of foot is dragging along cage | |
| bottom/table, walk is slightly wobbly, feet wider | |
| apart, occasionally throws out a leg to catch itself, | |
| or has “high-stepping” behavior, occasional | |
| stargazing behavior. | |
| Staggering or erratic walk, occasional circling behavior | 3 |
| frequent stargazing, loss of spatial sense or potential | |
| vision loss (running into cage-mates or walls), | |
| belly occasionally drags on ground, loss of balance. | |
| Endpoint phenotype. Mouse frequently falls over, | 4 |
| cannot control back limbs, belly frequently on | |
| ground, head and tail move erratically to keep | |
| balance. | |

Assessments are based on scoring developed at Jackson Laboratories, (www.jax.org/) during characterization of the Fxn ncKO mouse model.

### Example 3—rAAVhu68.hFXN Delivery in Nonhuman Primates

### A. IV Dose Ranging Pharmacology, Biodistribution, and Safety Study in Non-Human Primates (Nonclinical Study 7)

The purpose of this non-GLP-compliant study was to determine the pharmacokinetic, safety profile and transduction efficiency of cardiomyocytes, DRG sensory neurons and region of interest in the CNS (dentate nucleus), following IV administration of rAAVhu68.hFXN. NHPs were selected for this study as they replicate the CNS, PNS, and heart anatomy of the intended FDRA patient population. Adult NHPs (3-6 years old) were administered one of three doses of rAAVhu68.hFXN, 1.0×10¹³ GC/kg, 3.0×10¹³ GC/kg, or 1.0×10¹⁴ GC/kg. Age-matched NHPs were untreated and served as a control. In-life evaluations included daily clinical observations, physical exams, body weights, clinical pathology of the blood. Animals were necropsied 28 days post administration and a comprehensive list of tissues were harvested for histological evaluation and biodistribution. Histopathological examination was performed for the liver, heart, spinal cord, and DRG sensory neurons. Transgene expression was evaluated for the heart and DRG sensory neurons by in-situ hybridization (ISH) and further validated in the heart by immunohistochemistry (IHC).

rAAVhu68.hFXN related pathological findings included mild hepatocellular loss and individual cell apoptosis in portal areas with moderate chronic inflammation and secondary hepatic changes including minimal hepatocellular regeneration, bile duct hyperplasia and portal fibrosis. These findings were observed in NHPs administered the highest dose, 1.0×10¹⁴ GC/kg (½ animals). These liver findings, albeit more severe, have been reported in the literature associated with high dose IV AAV9 and AAV9-like vector administration with different transgenes and are likely not related to rAAVhu68.hFXN administration (Hinderer et al., 2018). Minimal myocardial infiltrates were observed in the heart in the majority of rAAVhu68.hFXN-treated NHPs. This finding has been reported as background findings in NHPs and are not considered rAAVhu68.hFXN treatment-related (Sato et al., 2012). No histopathological findings were observed in the DRG in any of the rAAVhu68.hFXN treated groups.

Dose-dependent transduction of the heart was observed in all rAAVhu68.hFXN treatment NHPs. Robust transgene expression in cardiomyocytes of the heart (data not shown) and DRG sensory neurons (data not shown) was observed in NHPs treated with doses of 3.0×10¹³ GC/kg and 1.0×10¹⁴ GC/kg. IHC staining revealed minimal transgene expression in the dentate nucleus and upper motor neurons (data not shown).

In Nonclinical study 7, safety and transduction efficiency of the heart and DRG was assessed after IV administration of rAAVhu68.hFXN at a dose of 1.0×10¹³ GC/kg, 3.0×10¹³ GC/kg, and 1.0×10¹⁴ GC/kg. Higher doses were not evaluated due to of risk of systemic toxicity in higher doses such as 2.0×10¹⁴ GC/kg (Hinderer et al., 2018). The study duration was 28 days, allowing peak transgene expression to be achieved. However systemic administration has poor transduction profile of deep brain regions such as the dentate nucleus, which is an important area leading to FRDA neuropathology. Nonclinical study 8 assessed the safety and transduction efficiency of unilaterally and bilaterally MRI-guided direct injection of rAAVhu68.hFXN into the deep cerebellar nuclei (DCN) via convection-enhanced delivery (CED) in two adult NHPs. The dose was chosen to ensure high transduction and assess the safety of introducing high levels of the transgene into the dentate nucleus. Due to the achieved transgene expression in Nonclinical Study 7 and results from Nonclinical Study 8, the toxicology studies includes both IV administration of rAAVhu68.hFXN (Nonclinical Study 11) and the dual ROA (Nonclinical Study 12). The IV dose(s) were chosen based on robust transgene expression of frataxin in the heart (Nonclinical Study 7) and the IDN dose chosen based on robust transgene expression in the dentate nucleus (Nonclinical Study 8). Study duration of the toxicology studies (Nonclinical Study 11 and Nonclinical Study 12) are 120 days to allow for a comprehensive assessment of safety and transgene expression in target organs.

rAAVhu68.hFXN at doses of 3.0×10¹³ GC/kg and 1.0×10¹⁴ GC/kg administered IV to NHPs lead to transduce of cardiomyocytes and DRG sensory neurons providing evidence for the potential of rAAVhu68.hFXN treatment to impact cardiac and PNS disease affected in FRDA patients. Minimal transduction of the dentate nucleus and upper motor neurons were observed suggesting IV administration of rAAVhu68.hFXN may not be the optimal ROA to target CNS tissues.

B. Distribution of AAV Serotype hu68 Vector in the Non-Human Primates (NHP) Dentate Nucleus of the Cerebellum (Nonclinical Study 8)

The purpose of this non-GLP-compliant study was to assess the safety and distribution of unilaterally and bilaterally MRI-guided direct injection of rAAVhu68.hFXN into the deep cerebellar nuclei (DCN) via convection-enhanced delivery (CED) in two adult (5-10 years old) NHPs. The devices used for DCN delivery is the same as in the Phase 1/2 FIH clinical trial (ClearPoint® System). The first animal was administered rAAVhu68.hFXN at a total dose of 1.71×10¹³ GC with a contrast agent (2 mM ProHance®, Gadoteridol) in a volume of 200 μl via a single (unilateral) transfrontal trajectory through the left hemisphere that targeted the middle of the DCN. The second animal was administered rAAVhu68.hFXN at a dose of 8.56×10¹² GC with a contrast agent (2 mM ProHance®, Gadoteridol) in a volume of 100 μl per site via two (bilateral) transfrontal trajectories (one per hemisphere) that targeted the left and right DCN. In both animals, a cannula was first inserted into the brain directing the injection of rAAVhu68.hFXN into the DCN which was confirmed via hyperintense signal on magnetic resonance imaging (MRI) emitted by the contrast agent (ProHance®, Gadoteridol). In-life evaluations included daily cage side observations of animal health and neurological symptoms. Animals were necropsied 30 days post administration and the body was transcardially perfused with cold phosphate buffered saline (PBS) with 0.026% (2.64 IU/mL) heparin. The entire brain was harvested, placed in a brain matrix and coronally sliced into 9-mm blocks. Coronal blocks were transferred into buffered formalin and processed for histology. Representative samples of brain (complete), liver, spleen, kidneys, lung, CSF, heart, testes, spinal cord (sections of cervical, thoracic and lumbar) were harvested and transferred into buffered formalin. Histopathological evaluation was performed on the brain and spinal cord and transgene expression in the brain evaluated by ISH.

The majority of microscopic findings in the forebrain, thalamus and medulla were considered likely procedural related as these findings were typically small discrete foci consistent with linear tracts (i.e. cannula/needle tract) resulting from direct injection into the brain. The microscopic findings included minimal to mild infiltrates of gitter cells along with other glial cells. There was no microscopic evidence of neuronal degeneration or necrosis. Cerebellar histopathology demonstrated minimal gliosis and parenchymal loss. Cerebellar findings consisted of multifocal to regional gliosis with or without reactive astrocytosis and perivascular mononuclear cell infiltrates with occasional perivascular edema. There were variably-sized discrete regions composed of gitter cells, similarly representing neural tissue injury likely secondary to DCN injection; however, there was no evidence of neuronal degeneration or necrosis. The surrounding affected white matter tracts exhibited similar axonal damage as described above. Given that this was the target site of the injection, it was not unexpected that the microscopic findings were slightly more severe. For the most part these findings were considered likely procedural related to the injection; however, test article associated perivascular mononuclear cells and edema, which were restricted to the DCN (target site), cannot be ruled out. The animals were behaviorally normal throughout the study with no associated neurologic deficits. Transgene expression in both animals was confined to the cerebellum (data not shown), with the most robust transgene expression occurring following bilateral IDN injections of rAAVhu68.hFXN (data not shown).

Robust and local transduction of the dentate nucleus with no dose limiting toxicity was observed following unilateral and bilateral IDN administration of rAAVhu68.hFXN leading the possibility to impact the neurological symptoms in FRDA patient.

### Example 4-Pharmacology and Toxicology Studies with rAAVhu68.hFXN

A. Efficacy of rAAVhu68.hFXN Following Intravenous Administration in Cardiac Conditional Knockout Fxn Mice to Determine the Minimum Effective Dose (MED) (Nonclinical Study 9)

This study evaluates the efficacy and determine the MED of rAAVhu68.hFXN following IV administration to Fxn cKO mice (Fxn^{flox/null}::Ckmm-Cre). The study is conducted per GLP regulations or with Quality Assurance (QA) oversight (conduct per protocol and oversight of key phases of the study and final report). Adult Fxn cKO mice (28 days old) receive a single IV administration of rAAVhu68.hFXN at one of four dose levels, 3.0×10¹² GC/kg, 1.0×10¹³ GC/kg, 3.0×10¹³ GC/kg, or 1.0×10¹⁴ GC/kg. Age-matched Fxn cKO and Fxn unaffected Cre mice (Fxn^{+/flox}::Ckmm-Cre) are administered vehicle (PBS) as controls. The age of the animals was selected to mimic the proposed clinical trial population. The selected doses are based on results from Nonclinical Study 2 and Nonclinical Study 3. In-life assessments include daily viability checks when animal are 7 weeks of age (˜49 days of age), weekly body weight measurements, and survival. Mice are necropsied 120 days after administration or when animals reach the prespecified euthanasia endpoint (determined by veterinarian or by 20% weight loss from maximal weight). At necropsy, blood are collected for complete blood counts (CBCs) and serum clinical chemistries as toxicology readouts, and for measurement of cardiac stress marker (GDF-15) as pharmacology endpoint. A complete tissue list are harvested for comprehensive histopathology evaluation. Heart tissue are harvested to evaluate disease-relevant biomarker of mitochondrial function (SDH Activity) and cardiomyocyte transduction are quantified by ISH or IHC.

B. Efficacy of rAAVhu68.hFXN Following Intravenous Administration in Neurological Conditional Knockout Fxn Mice to Determine the Minimum Effective Dose (MED) (Nonclinical Study 10)

This study evaluates the efficacy and determine the MED of rAAVhu68.hFXN following IV administration to Fxn ncKO mice. The study is conducted per GLP regulations or with QA oversight (conducted per protocol and oversight of key phases of the study and final report). Adult Fxn ncKO mice (28 days old) receive a single IV administration of rAAVhu68.hFXN at one of four dose levels, 3.0×10¹² GC/kg, 1.0×10¹³ GC/kg, 3.0×10¹³ GC/kg, 1.0×10¹⁴ GC/kg, or vehicle (PBS). Age-matched Fxn unaffected mice mice are administered vehicle (PBS) as a control. The age of the animals was selected to mimic the proposed clinical trial population. The selected doses are based on results from Nonclinical Study 6 and Nonclinical Study 3. In-life assessments include daily viability checks, weekly neurological assessment (Neuroscore, See Nonclinical Study 6 and Table 5 for details of the assessment), and body weight measurements, monthly neuromotor function (RotaRod, See Nonclinical Study 6 for details of the assessment) starting at 56 days of age (8 weeks of age) and survival. Mice are necropsied 120 days after administration or when animals reach the prespecified euthanasia endpoint (determined by veterinarian when mice reach 20% weight loss from maximal weight or a Neuroscore of 4). At necropsy, blood is collected for complete blood counts (CBCs) and serum clinical chemistries and tissues are harvested for comprehensive histopathology evaluation.

C. Toxicology of rAAVhu68.hFXN Administered Intravenous in Adult Rhesus Macaques (Nonclinical Study 11)

A 120 day GLP-compliant safety study is conducted in adult rhesus macaques (3-8 years of age) to investigate the toxicology of rAAVhu68.hFXN following IV administration. Rhesus macaques receive one of three dose levels of rAAVhu68.hFXN: 1.0×10¹³ GC/kg, 3.0×10¹³ GC/kg, or 1.0×10¹⁴ GC/kg (Table 6). Additional adult NHPs are administered vehicle, intrathecal final formulation buffer (ITFFB) as a control. The rAAVhu68.hFXN dose levels selected are equivalent to three highest doses that are evaluated in the MED studies (Nonclinical Study 9 and Nonclinical Study 10). The 120 day evaluation period was selected to allow sufficient time for a transgene product to reach stable plateau levels following IV AAV administration. The age of administration was selected to be representative of the proposed clinical trial population in terms of anatomy.

TABLE 6
Rhesus Macaque GLP Toxicology Study.

| | Group Designation | | | |
|---|---|---|---|---|
| | 1 | 2 | 3 | 4 |
| Number of | 2 | 3 | 3 | 3 |
| Macaques | | | | |
| Sex | M + F | M + F | M + F | M + F |
| Age | Adult (3-8 | Adult (3-6 | Adult (3-6 | Adult (3-6 |
| | years) | years) | years) | years) |
| Test Article | Vehicle (PBS) | rAAVhu68.hFXN | rAAVhu68.hFXN | rAAVhu68.hFXN |
| ROA | IV | IV | IV | IV |
| Vector Dose | NA | 1.0 × 10¹³ GC/kg | 3.0 × 10¹³ GC/kg | 1.0 × 10¹⁴ GC/kg |
| Necropsy Day | 120 | 120 | 120 | 120 |

Abbreviations: F, female; GC, genome copies; GLP, good laboratory practice; ITFFB, intrathecal final formulation buffer; IV, intravenous; M, male; NA, not applicable; ROA, route of administration.

Baseline neurologic examinations, clinical pathology (cell counts with differentials, clinical chemistries, and a coagulation panel), CSF chemistry, and CSF cytology are performed. After rAAVhu68.hFXN or vehicle administration, the animals are monitored daily for signs of distress and abnormal behavior. Neurological assessments are divided into five sections evaluating the following: mentation, posture and gait, proprioception, cranial nerves, and spinal reflexes. The tests for each assessment are performed in the same order each time. Assessors are not formally blinded to the treatment group; however, assessors typically remain unaware of treatment group at the time of assessment. Numerical scores are given for each assessment category as applicable and are recorded (normal: 1; abnormal: 2; decreased: 3; increased: 4; none: 5; N/A: not applicable).

Blood and CSF clinical pathology assessments and neurologic examinations are performed on a weekly basis for 30 days following rAAVhu68.hFXN or vehicle administration, and every 30 days thereafter. Additional blood clinical pathology assessment are performed 3 days after administration. At baseline, study days 0, 30 and 120, anti-AAVhu68 NAbs and cytotoxic T lymphocyte (CTL) responses to AAVhu68 and the rAAVhu68.hFXN transgene product are assessed by an interferon gamma (IFN-γ) enzyme-linked immunospot (ELISpot) assay. Nerve conduction velocity (NCV) assessment is performed at baseline, study days 14, 30 and 120. Briefly, NCV assessments are performed on sedated NHPs. The stimulator probe is positioned over the median nerve with the cathode closest to the recording site and two needle electrodes are inserted subcutaneously on digit II at the level of the distal phalanx (reference electrode) and proximal phalanx (recording electrode), while the ground electrode is placed proximal to the stimulating probe (cathode). Following determination of the optimal stimulus location the stimulus strength is progressively increased and the stimulus responses are recorded and averaged. Responses are averaged, the distance (cm) from the recording site to the stimulation cathode are measured and the conduction velocity is calculated using the onset latency of the response and the distance (cm). Both the conduction velocity and the average of the SNAP amplitude are reported. The median nerve are tested bilaterally.

Echocardiogram assessment is performed at baseline, study days 14, 30, 60, 90 and 120. Animals are necropsied 120 days after administration and tissues harvested for biodistribution and a comprehensive histopathological examination. Additional specialized staining is performed for the heart. Cardiac and DRG transgene expression are evaluated with IHC or ISH. In addition, lymphocytes are harvested from the circulating compartment (peripheral blood mononuclear cells), spleen, and liver to evaluate the presence of T cells reactive to both the capsid and transgene product in these organs at the time of necropsy. Tissues are collected for vector biodistribution. Urine and feces are collected for vector excretion analysis using qPCR. The CSF and serum are also collected and archived for future possible analysis.

D. Toxicology of rAAVhu68.hFXN Administered Intravenous and Intraparenchymal (Dentate Nucleus) in Adult Rhesus Macaques (Nonclinical Study 12)

A 120 day safety study is conducted in adult rhesus macaques (3-8 years of age) to investigate the toxicology of rAAVhu68.hFXN following dual ROA, IV and IDN administration. Rhesus macaques receive an IV administration of rAAVhu68.hFXN at a dose of 3.0×10¹³ GC/kg and a bilateral IDN injection of rAAVhu68.hFXN at a dose of 1.5×10¹² GC in 50 μL of ITFFB per injection for a total IDN dose of 3.0×10¹² GC/kg. The IV and IDN doses are based on the Nonclinical Study 7 and Nonclinical Study 8. The devices used for IDN administration is the same as in the Phase 1/2 FIH clinical trial (ClearPoint® System). The dosing regimen matches the Phase 1/2 FIH clinical trial (i.e., IV infusion followed by IDN injection). The 120 day evaluation period was selected to allow sufficient time for a transgene product to reach stable plateau levels following IV and IDN AAV administration. The age of administration was selected to be representative of the proposed clinical trial population. The study has QA oversight (conducted per protocol and oversight of key phases of the study and final report).

Baseline neurologic examinations (see Nonclinical Study 11 for details), clinical pathology (cell counts with differentials, clinical chemistries, and a coagulation panel), CSF chemistry, and CSF cytology are performed. After rAAVhu68.hFXN or vehicle administration, the animals are monitored daily for signs of distress and abnormal behavior.

Blood and CSF clinical pathology assessments and neurologic examinations are performed on a weekly basis for 30 days following rAAVhu68.hFXN or vehicle administration, and every 30 days thereafter. Additional blood clinical pathology assessment are performed 3 days after vector administration. At baseline, study days 0, 30 and 120, anti-AAVhu68 NAbs and cytotoxic T lymphocyte (CTL) responses to AAVhu68 and the rAAVhu68.hFXN transgene product are assessed by an interferon gamma (IFN-γ) enzyme-linked immunospot (ELISpot) assay. Nerve conduction velocity (NCV) assessment (see Nonclinical Study 11 for details) is performed at baseline, study days 14, 30 and 120. Echocardiogram assessments are performed at baseline, study days, 30, 60, 90 and 120.

Animals are necropsied 120 days after administration and tissues harvested for biodistribution and a comprehensive histopathological examination. In addition, lymphocytes are harvested from the circulating compartment (peripheral blood mononuclear cells), spleen, and liver to evaluate the presence of T cells reactive to both the capsid and transgene product in these organs at the time of necropsy. Tissues are collected for vector biodistribution. Urine and feces are collected for vector excretion analysis using qPCR. The CSF and serum are also be collected and archived for future possible analysis in case any finding warrants analysis.

### Example 5—Cardiac and CNS Co-Administration of rAAVhu68.hFXN

Stocks of rAAVhu68.hFXN were formulated for intravenous delivery designed for targeting cardiomyocytes and DRG neurons and/or co-administration with rAAVhu68.hFXN formulated for direct injection to target dentate nuclei.

rAAVhu68.hFXN administration increased heart frataxin levels and significantly improved survival. Studies in nonhuman primates have demonstrated that the rAAVhu68.hFXN can efficiently express frataxin in key cellular targets with an acceptable safety profile. The IV ROA for rAAVhu68.hFXN was chosen to provide increased frataxin levels to the heart to address the cardiac manifestations of the disease, as cardiac failure is the cause of death for the majority of the population under study (early-onset FRDA patients). IDN administration for rAAVhu68.hFXN was chosen to increase frataxin level locally in the dentate nucleus to address the ataxic symptoms and prevent further impairment of speech, swallowing and gait in FRDA patients. In certain embodiments, rAAVhu68.hFXN may be suitable for combined central and intravenous routes of administration to address the cardiac and neurological features of FRDA.

### Example 6—Method for Dose Scaling Between Species

### A. Dose Scaling

The Fxn conditional murine disease models are utilized to demonstrate pharmacology of rAAVhu68.hFXN cannot be used to directly determine doses for human studies because of differences in target cells, ROA, and transduction differences between species. To determine the dose range for the Phase 1/2 FIH trial (Example 7), transduction data from the NHP toxicology studies is utilized. For the vector dose administered IV, the minimal effective dose (MED) is informed by data from the murine pharmacology study as well as the NHP toxicology study. The pharmacology study in the Fxn cKO mouse model is used to determine the relationship between the percentage of cardiomyocytes transduced and rescue of functional endpoints (e.g., a significant increase in survival). The NHP toxicology studies are then be utilized to identify a vector dose that achieves a similar level of cardiomyocyte transduction. The dose identified in NHP that yields a similar level of cardiomyocyte transduction to the minimum level associated with significant functional improvements in mice is considered the MED for the IV dose. The IV dose is scaled from NHPs to humans based on body mass.

For the vector dose administered to the dentate nuclei, a MED is determined in NHP based on transduction of target large neurons of the dentate nuclei. For scaling to human doses, the vector concentration is the same as that utilized in NHPs, but the total volume (and total vector dose) is linearly scaled based on the average relative volume of the dentate nuclei in NHPs and humans. Proportionally increasing the injection volume while maintaining a constant vector concentration allows the infusion to cover the entire dentate nucleus of each species while maintaining similar vector exposure to target cells.

### B. Neuron Toxicity

Minimal to mild asymptomatic degeneration of DRG sensory neurons is not expected to appear in the rAAVhu68.hFXN GLP NHP toxicology study at all doses evaluated. However, the true risk of sensory neuron toxicity in humans is unknown. The current trial is designed to further improve on the safety profile of previous AAV clinical trials by using a dual ROA that requires lower doses of vector than those typically administered systemically, which appears to result in a lower degree of sensory neuron toxicity. This study also employs detailed monitoring for sensory changes as well as nerve conduction studies to detect even subclinical DRG toxicity. Given the severity of FRDA, the risk-benefit profile for dual route administration of rAAVhu68.hFXN is expected to remain favorable despite the unknown risk of sensory neuron toxicity.

### C. Cardiotoxicity Potential of Frantaxin Overexpression

FRDA is caused by mutations in the FXN gene encoding the frataxin protein leading to a lack of frataxin and accumulation of iron in the mitochondria which predominantly affects cardiomyocytes and defined neuron populations in the CNS. rAAVhu68.hFXN is being developed to target peripheral organs, most notably cardiac myocytes (IV ROA) and central organs, cerebellum and sensory DRG neurons (IDN ROA), leading to supra-physiological levels of frataxin within days of administration. A recent publication explored the possibility of cardiotoxicity due to frataxin overexpression after gene therapy in Fxn conditional knockout (Mck) mice compared to wild type mice using two different vectors (non-optimized and optimized; (Belbellaa et al., 2020)). The results from the three studies presented in the paper showed cardiotoxicity when frataxin is expressed at >20-fold the endogenous level but lack of cardiotoxicity when expressed at endogenous levels <9-fold. However, transgene expression and cardiotoxicity seemed to be higher when there was no frataxin present than if frataxin was already expressed.

The relevance of these results to our gene therapy program is unclear. Cardiotoxicity evaluation in NHP is more relevant to the proposed Phase 1/2 clinical trial than cardiotoxicity evaluation in mice. Cardiotoxicity was not observed in the completed NHP study (Nonclinical Study 7) when rAAVhu68.hFXN was administered IV at doses up to 1.0×10¹⁴ GC/kg. However, this study was only 28 days in duration which may be too short to observe the hypertrophic cardiomyopathy as was reported and echocardiograms were not performed thus it is unclear if hypertrophic cardiomyopathy was present.

To evaluate possible cardiotoxicity due to frataxin overexpression after IV administration, echocardiogram assessments are conducted at different timepoints in the NHP toxicology studies (Nonclinical Study 11 and Nonclinical Study 12). In addition, following necropsy, a full histopathology evaluation of the heart is performed, while also assessing transgene expression in the heart. These assessments assist with the evaluation of co-localization of cardiac pathology and frataxin overexpression. Furthermore, in the MED study (Nonclinical Study 9), we perform full histopathology evaluation, and also evaluate SDH activity in the heart, since it was reported to be impaired due to frataxin overexpression.

Cardiotoxicity was apparent 8-14 weeks after gene therapy delivery. The proposed 120 day duration of the MED study (Nonclinical Study 9) and toxicology studies (Nonclinical Study 11 and Nonclinical Study 12) are sufficient duration to assess cardiotoxicity.

### Example 7—Clinical Protocol

A. Manufacturing process for rAAVhu68.hFXN

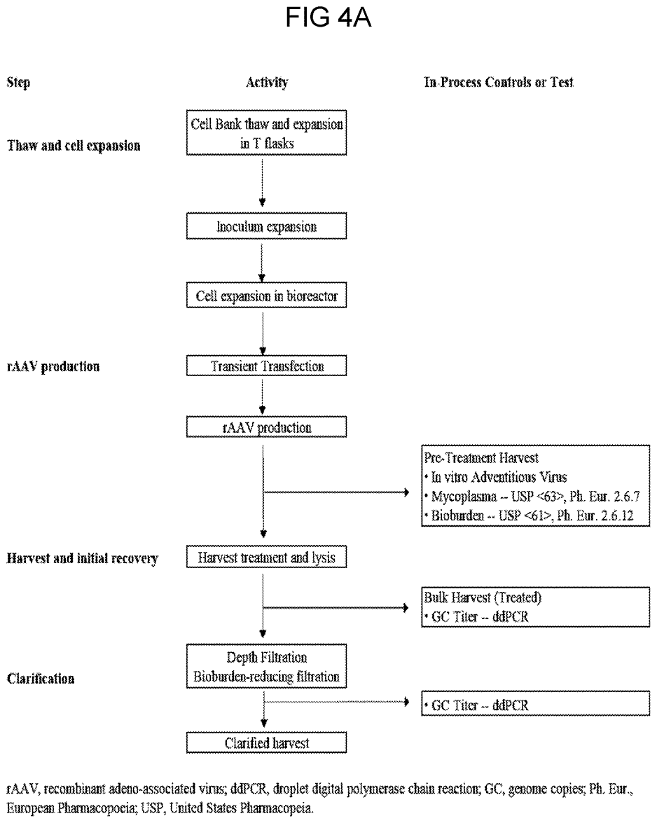

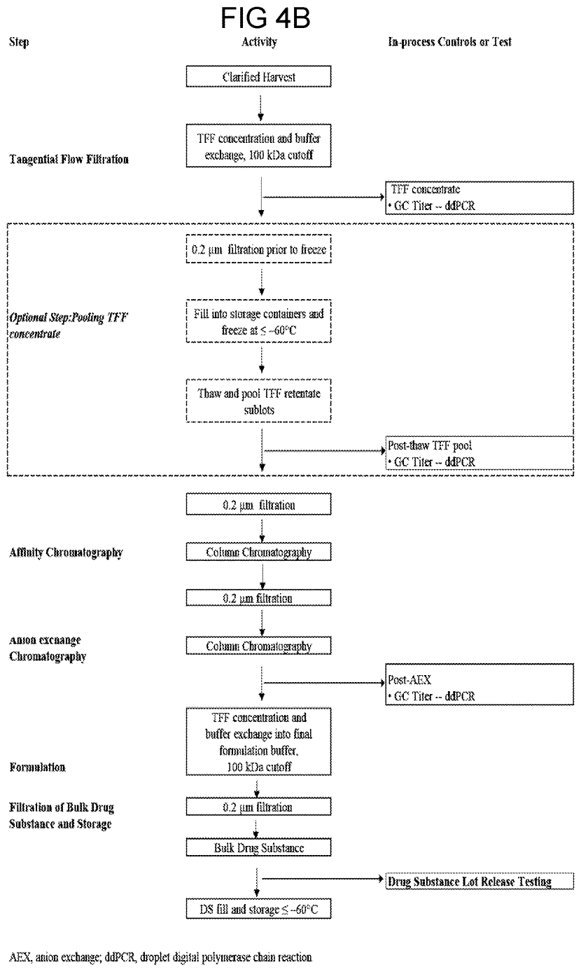

rAAVhu68.hFXN for the FIH trial is manufactured by transient transfection of HEK293 cells followed by downstream purification using a process previously developed. The product is produced at the CMO facility in a controlled environment consistent with FDA regulations (“Guidance for Industry-cGMP for Phase 1 Investigational Drugs,” July 2008), which ensures the safety, identity, quality, purity, and strength of the manufactured biologic. A manufacturing process flow diagram is shown in FIGS. 4A and 4B. The proposed in-process tests are depicted on the right side of the diagram. A description of each production and purification step is also provided. Product manufacturing follows a linear flow of unit operations and utilizes disposable, closed bioprocessing systems unless otherwise specified. rAAVhu68.hFXN is the sole product manufactured within a specified production suite at a single time at the multiproduct CMO, with changeover controls in place between products. Cells are thawed in single use T-flasks and expanded into single use sterile shake flasks that are used to innoculate a fixed bed, controlled, single use production bioreactor. All seed train open manipulations are performed in class II biological safety cabinets (BSCs) in an ISO Class 5 environment. The purification process is performed using commercially supplied filters and chromatography resins. The Intrathecal Final Formulation Buffer (ITFFB) solution is manufactured by process comprising compounding of the ITFFB formulation buffer, sterile filtration, followed by aseptic filing into vials. The manufacturing process follows standard procedures for solution compounding and 0.2 μm sterile filtration followed by aseptic processing.

### B. Overview of First-In-Human Trial

The FIH trial is a Phase 1/2, open-label, multi-center, dose escalation study of rAAVhu68.hFXN to evaluate safety, tolerability, pharmacodynamics, and efficacy in subjects with early onset Friedreich's Ataxia (FRDA) aged 16 years and older. A two-stage dosing design is utilized. Dosing in each stage and cohort consists of a one-time administration of two doses of rAAVhu68.hFXN, each delivered via a different route within 24 hours. The first dose is administered via intravenous (IV) infusion followed by a second dose administered to each of the dentate nuclei via intraparenchymal dentate nucleus (IDN) injection. Stage 1 comprises the dose escalation phase of the study and involves sequential administration of a low dose of dually-delivered rAAVhu68.hFXN (Cohort 1) followed by a high dose of dually-delivered rAAVhu68.hFXN (Cohort 2) in non-ambulatory subjects. Both dose levels have the potential to confer therapeutic benefit, with the understanding that, if tolerated, the higher dose regimen is expected to be advantageous. The sequential evaluation of the low dose regimen (Cohort 1) followed by the high dose (Cohort 2) enables the identification of the maximum tolerated dose (MTD) of the two dose regimens tested. Based on Stage 1 data, ambulatory FRDA subjects (Cohort 3) are enrolled into Stage 2 and are dosed with the MTD of rAAVhu68.hFXN in a parallel fashion. The rAAVhu68.hFXN dose levels is determined based on data from the murine MED study (Examples 4A and 4B) and GLP NHP toxicology study (Examples 4C and 4D) and consists of a low dose (administered to Cohort 1) and a high dose (administered Cohort 2). Our standard approach is that a safety margin is applied so that the high dose selected for human subjects is 30-50% of the equivalent MTD in NHPs. The low dose is typically 2-3-fold less than the selected high dose provided it is a dose that exceeds the equivalent scaled MED in the animal studies.

The aim of the study is to evaluate the safety and tolerability of rAAVhu68.hFXN. Additionally, pharmacodynamic outcomes, as well as exploratory efficacy outcomes, are assessed to evaluate the potential of rAAVhu68.hFXN to improve or stabilize the symptoms of FRDA.

The study design staggers enrollment of each subject by a 30 day interval in both Cohort 1 (low dose) and Cohort 2 (high dose). The rationale for this approach is that the delivery method for rAAVhu68.hFXN is a novel procedure and this approach allows for additional safety monitoring after each subject undergoes the procedure. Furthermore, this 30-day window captures the time when maximal gene expression is expected based on nonclinical data.

An independent Data Safety Monitoring Board (DSMB) conducts a safety review of all accumulated safety data between cohorts and after full enrollment of the second cohort to make a recommendation regarding further conduct of the trial. The DSMB also conducts a review any time a safety review trigger (SRT) is observed. The 30-day dosing interval between each subject in Cohorts 1 and 2 allows for evaluation of AEs indicative of acute immune reactions, immunogenicity, or other dose-limiting toxicities during the interval in which maximal gene expression is expected. The DSMB review conducted at the end of Cohorts 1 and 2 is performed once 30 days of safety data from each patient in the respective cohort has been collected and fully analyzed. This data collection is expedited by the fact that the planned trial is open label and therefore data can be analyzed in real time. If there is a safety event that triggers a DSMB review outside of the specified checkpoints, this interval may be extended such that no new recruitment occurs until after a decision is made by the DSMB. The 30-day interval may also be extended to accommodate this review.

Provided that the DSMB recommends study continuation after completion of Cohort 2, additional subjects are enrolled in an expansion cohort that receive the MTD. Enrollment of these additional subjects does not require a 30-day observation window between subjects.

All treated subjects are followed for 2 years to evaluate the safety profile and characterize the pharmacodynamic and efficacy properties of rAAVhu68.hFXN. Subjects are followed for an additional 3 years (for a total of 5 years post-dose) during the LTFU period of the study to evaluate long-term clinical outcomes, which is in line with the draft “FDA Guidance for Industry: Long Term Follow-Up after Administration of Human Gene Therapy Products” (January 2020).

TABLE 7
First-in-Human clinical Trial Protocol Synopsis.

| | |
|---|---|
| Protocol(s) | A Phase 1/2 Multi-Center, Dose Escalation Study to Assess the Safety, Tolerability, |
| Title | and Pharmacodynamics of Single Doses of Dual-Administration GTP-212 |
| | Delivered Intravenously (IV) and via Intraparenchymal Dentate Nucleus (IDN) |
| | injection of Subjects with Friedreich's Ataxia (FRDA) |
| Objectives and | The primary objective of this study is to assess the safety and tolerability of |
| Endpoints | a dual route administration of rAAVhu68.hFXN through evaluation of: |
| | Adverse events (AEs) and serious adverse events (SAEs) |
| | Vital signs and physical examinations |
| | Neurological examinations |
| | Cardiac monitoring |
| | Laboratory assessments (serum chemistry, hematology, coagulation |
| | studies, liver function tests [LFTs], urinalysis, and CSF chemistry |
| | and cytology) |
| | C-SSRS |
| | Immunogenicity of the vector and transgene product |
| | Vector shedding |
| | The secondary objectives of this study are to assess the pharmacodynamics |
| | and biological activity and efficacy of dual administration of |
| | rAAVhu68.hFXN as well as improvements in the quality of life of treated |
| | subjects over 2 years based on the following endpoints: |
| | Quality of Life (QoL) as assessed by the Activities of Daily Living |
| | (ADL) questionnaire at 2 years post-rAAVhu68.hFXN treatment |
| | Quality of Life (QoL) as assessed by the Patient- and Clinician- |
| | Global Impression of Change surveys (P-GIC and C-GIC, |
| | respectively) at 2 years post-rAAVhu68.hFXN treatment |
| | Change from baseline in ejection fraction assessed at the 2 years |
| | post-rAA Vhu68.hFXN treatment |
| | Heart strain and relative wall thickness (RWT) as measured by |
| | echocardiogram and cardiac MRI at 2 years post-rAAVhu68.hFXN |
| | treatment |
| | Further secondary key efficacy endpoints include: |
| | For Stage 1 (Cohorts 1 and 2): |
| | Change in speech ability, as assessed by a summative speech |
| | assessment score measuring multiple aspects of speech and voice |
| | quality at 2 years post-rAAVhu68.hFXN treatment |
| | For Stage 2 (Cohort 3): |
| | Ambulation as measured by change from baseline at the 2 year time |
| | point in the upright stability subsection of the mFARS assessment |
| | The exploratory objectives of the study are to further assess the efficacy of |
| | dual route administration of rAAVhu68.hFXN through the following |
| | endpoints: |
| | General: |
| | Survival |
| | Levels of frataxin expression in serum samples evaluated at |
| | 1year post-rAAVhu68.hFXN treatment |
| | Quality of Life (QoL) as assessed by a FRDA-specific |
| | patient reported outcome questionnaire currently being |
| | developed by the Friedreich's Ataxia Research Alliance at 2 |
| | years post-rAAVhu68.hFXN treatment |
| | Neurological Endpoints |
| | mFARS assessment as compared to baseline score to |
| | measure overall disease state over time |
| | 9-hole peg test (9HPT) if subject is able to perform in <5 |
| | minutes; or use a spoon dexterity test if subject is unable to |
| | complete the 9HPT |
| | Further exploratory neurological endpoints for subjects |
| | enrolled into Stage 2 only include: |
| | Ambulation as assessed by a 25-foot walk test at 2 |
| | years post-rAAVhu68.hFXN treatment |
| | Cardiac Endpoints |
| | Electrocardiogram changes |
| | Absence of progression of cardiac symptoms, including ICD |
| | and heart failure hospitalization |
| Study Design | This is a Phase 1/2 first-in-human, multi-center, dose escalation study to evaluate |
| | safety, tolerability, pharmacodynamics and efficacy of rAAVhu68.hFXN in up to 16 |
| | subjects, but at least 12 subjects with early onset FRDA with age of onset at <14 |
| | years old and current age 16 years old or older. The study provides proof of concept |
| | in assessing safety, tolerability, pharmacodynamics and exploratory efficacy of |
| | dual-route administration of rAAVhu68.hFXN, all of which assist in defining |
| | rAAVhu68.hFXN dose and endpoints for a registration trial. |
| | A two-stage dosing design, comprising 3 cohorts (described below), are utilized. |
| | Patients are administered corticosteroids immediately prior to and for 1-week post |
| | IDN administration to minimize any potential brain inflammation related to the |
| | study procedure. Furthermore, if at any time post-IV/IDN administration |
| | transaminase elevations are observed, the steroid dose are increased or reinitiated. A |
| | prolonged regimen of corticosteroids may also be utilized in order to mitigate |
| | potential immune-related injury, such as hepatoxicity. The final prophylactic steroid |
| | protocol is developed in line with GLP toxicology studies to be performed. In line |
| | with this corticosteroid administration, subjects are closely monitored for signs of |
| | brain inflammation and immune toxicity as well as issues related to steroid dosing. |
| | A full dosing regimen is provided in the eventual first-in-human study protocol. |
| | Dosing in each stage and cohort consist of a one-time administration of two doses of |
| | rAAVhu68.hFXN with the first dose delivered via the IV ROA followed by a |
| | second dose delivered via an IDN injection within 24 hours. This administration |
| | order is subsequently referred to as dual-route dosing. Dose administration occurs |
| | in an inpatient setting. Each patient is seen on a regular basis for completion of |
| | study-related procedures as listed in the Schedule of Events. An independent Data |
| | Safety Monitoring Board (DSMB) is utilized throughout the study. The DSMB |
| | specifically advises on continuation of dose escalation within Stage 1 of the study as |
| | well as provide continual safety monitoring oversight any time a safety review |
| | trigger (SRT) is observed. |
| | Stage 1 (n = 6) |
| | Stage 1 of this study is the dose escalation phase and assesses the safety and |
| | tolerability of a one-time, dual-route administration of rAAVhu68.hFXN in 2 |
| | treatment cohorts (Cohort 1 and Cohort 2). Each cohort consists of 3 non- |
| | ambulatory FRDA subjects. Sequential dosing, with a 30-day dosing interval |
| | between each subject in each cohort, is utilized to assess adverse events (AEs) |
| | indicative of acute immune reactions, immunogenicity, and other dose-limiting |
| | toxicities. Both dose levels evaluated in Stage 1 are anticipated to confer therapeutic |
| | benefit, with the understanding that, if tolerated, the higher dose is expected to be |
| | advantageous. |
| | Cohort 1 (low dose rAAVhu68.hFXN): This cohort consists of 3 subjects (Subjects |
| | 1-3) who receive dual route administration of rAAVhu68.hFXN with both |
| | procedures being performed within 24 hours. Dosing in this cohort is sequential, |
| | with each subject dosing separated by 30 days. All available Cohort 1 safety data |
| | are evaluated by an DSMB 30 days after Subject 3 is administered |
| | rAAVhu68.hFXN. Based on their review, the DSMB provide a recommendation on |
| | the initiation of Cohort 2 dosing. |
| | Cohort 2 (high dose rAAVhu68.hFXN): This cohort consists of 3 new subjects |
| | (Subjects 4-6) who receive dual route administration of rAAVhu68.hFXN with both |
| | procedures being performed within 24 hours. Dosing in this cohort is sequential, |
| | with each subject dosing separated by 30 days. All available Cohort 2 safety data |
| | are evaluated by the DSMB 30 days after Subject 6 is administered |
| | rAAVhu68.hFXN to make a recommendation on study continuation. |
| | At this time, the DSMB also performs a combined review of all available Cohort 1 |
| | and Cohort 2 safety data to enable the identification of the maximum tolerated dose |
| | (MTD) to be evaluated in Stage 2 of this study. |
| | Stage 2 (n = 6 to 10) |
| | Stage 2 assesses the safety, tolerability, pharmacodynamics and efficacy of a one- |
| | time, dual-route administration of rAAVhu68.hFXN (with both procedures |
| | performed within 24 hours) in a single Cohort (Cohort 3) comprised of at least 6 |
| | (and up to 10) ambulatory FRDA subjects. All Cohort 3 subjects are dosed in |
| | parallel with the rAAVhu68.hFXN selected for evaluation based on the Stage 1 data |
| | review. |
| Study | The duration of participation in this protocol is 5 years in accordance with “FDA |
| Duration | Guidance for Industry: Long Term Follow-Up after Administration of Human Gene |
| | Therapy Products” (January 2020). |
| Number of | Up to 16 FRDA subjects are enrolled as follows: |
| Subjects | 6 non-ambulatory FRDA subjects; 3 in each of the Stage 1 cohorts (Cohorts |
| | 1 and 2) |
| | 6 to 10 ambulatory FRDA subjects in the Stage 2 cohort (Cohort 3) |
| Study Center | Multi-center |
| | Centers in the US and outside of the US capable of performing |
| | administration of gene product serve as rAAVhu68.hFXN dosing centers for |
| | all enrolled study participants |
| | Study follow-up visits and assessments are performed at select regional |
| | medical centers that are a part of the Friedreich's Ataxia Research Alliance |
| | (FARA) network in the US and EU by physicians who treat FRDA patients |
| Main Inclusion | All subjects are required to meet the following criteria: |
| Criteria | Age ≥16 years |
| | Genetically confirmed FRDA with age of onset <14 years |
| | Men and women of child-bearing potential (WCBP) must use a highly |
| | effective method of birth control defined as those, alone or in combination, |
| | that result in a low failure rate, i.e, less than 1% per year when used |
| | consistently and correctly. |
| | A baseline serum AAVhu68 neutralizing antibody titer ≤1:10 |
| | Subjects must be able to comprehend and be willing to provide an |
| | institutional review board/ethics committee (IRB/EC) approved Informed |
| | Consent Form (ICF) either themselves if over the age of 18 or with assent |
| | from the patient and an ICF signed by a parent or legal guardian if ages 16- |
| | <18. |
| | Subjects must be willing to comply with all study-related procedures and be |
| | available for the duration of the study. |
| | Additional Criteria for Stage 1 (Cohorts 1 and 2): |
| | Subjects are required to have lost ambulation, defined as having a |
| | Functional Staging of Ataxia score of either 5.0 or 5.5 (out of 6) |
| | Subjects are required to have some level of spontaneous speech, defined as a |
| | score of <3 (out of 3) on the Spontaneous speech mFARS sub-assessment |
| | Subjects meeting any of the following structural cardiac criteria: |
| | Left ventricular end diastolic diameter (LVEDD) > ULN |
| | Septal wall thickness (SWT) > ULN found on baseline ECHO/cMRI |
| | findings |
| | LV mass > ULN on baseline ECHO/cMRI findings |
| | Additional criteria for Stage 2 (Cohort 3): |
| | Ability to walk independently, with or without an assistive device, as |
| | defined by having the ability to both: |
| | Stand with feet apart and eyes open for >1 minute |
| | Complete a 25-foot walk test in <1 minute |
| | Subject has an mFARS score of >30 at baseline |
| | Subject has a septal wall thickness OR left ventricular (LV) mass >ULN on |
| | baseline ECHO/cMRI findings |
| Main | The following exclusion criteria apply to subjects in both study Stages: |
| Exclusion | Subjects who are compound heterozygous with 1154F or G130V point |
| Criteria | mutations for FRDA |
| | Subjects with severe dysarthria who would, in the opinion of the |
| | investigator, be unable to perform the speech related tasks |
| | Patients with vision worse than 20/200 or deaf patients at baseline |
| | Active arrhythmia OR ejection fraction <35% at baseline |
| | Diagnosed coronary heart disease |
| | Diagnosed symptomatic heart failure |
| | Uncontrolled diabetes defined as HbAlc >8% at baseline |
| | Any contraindication to the direct injection administration procedure into |
| | the dentate nucleus, including contraindications to fluoroscopic imaging |
| | Any contraindication to MRI or lumbar puncture (LP) |
| | Chronic renal insufficiency defined as estimated GFR <30 mL/min/1.73 m² |
| | Abnormal liver function tests (LFTs) at screening (AST or ALT >2 × upper |
| | limit of normal (ULN) and/or total bilirubin of >1.5 × ULN unless subject |
| | has unconjugated hyperbilirubinemia due to Gilbert's syndrome). |
| | History of cirrhosis or chronic liver disease based on documented |
| | histological evaluation or non-invasive imaging or testing. |
| | Patients with a positive test result for human immunodeficiency virus (HIV) |
| | or untreated Hepatitis C (HepC) |
| | Active tuberculosis, systemic fungal disease, or other chronic infection. |
| | Any clinically significant neurocognitive deficit not attributable to FRDA |
| | that may, in the opinion of the Investigator, confound interpretation of study |
| | results |
| | Any current or previous condition or physical exam or laboratory test |
| | finding that, in the opinion of the Investigator, would put the subject at |
| | undue risk or would interfere with evaluation and interpretation of the |
| | investigational product safety or efficacy results |
| | Enrollment in any other clinical study with an investigational product within |
| | 4 weeks prior to screening or within 5 half-lives of the investigational |
| | product used in that clinical study, whichever is longer, or any subject who |
| | has had any other prior gene therapy. |
| Investigational | rAAVhu68.hFXN |
| Product | |
| Reference | None |
| Therapy | |
| Route of | rAAVhu68.hFXN is administered via 2 routes (dual-route injection) as two |
| Administration | sequential doses to in-patient participants: subjects receive one dose via a peripheral |
| and Procedure | vein by IV infusion and the other by IDN injection. The volume of the IV infusion |
| | depends on the dose level and the weight of the subject. |
| Safety | Safety assessments, including collection of AEs and SAEs, physical and neurologic |
| Assessments | examinations, vital signs, clinical laboratory tests (serum chemistry, hematology, |
| | coagulation, LFTs, urinalysis), cardiac parameters, nerve conduction studies, and |
| | CSF cytology and chemistry (cell counts, protein, glucose) are performed at the |
| | times indicated in the study schedule). |
| | Additional safety assessments are added to the clinical trial protocol based on the |
| | findings of the GLP toxicology study. For instance, minimal to mild asymptomatic |
| | degeneration of DRG sensory neurons has been observed in NHP studies using |
| | AAV gene products (Hordeaux et al., 2018a; Hordeaux et al., 2018b; Hordeaux et |
| | al., 2018c) and is a potential risk for gene therapy products in general. It is not |
| | expected that this DRG toxicity to be observed for this program as it does not utilize |
| | an intracisterna magna route of administration and both doses for this trial are less |
| | than the dose at which this toxicity was observed using an IV administration |
| | (Hordeaux et al,, 2018c). Furthermore, based on existing non-clinical and clinical |
| | data for other AAV programs, it is anticipated that these sensory neuron findings do |
| | not translate to AEs in humans. However, if this DRG toxicity is observed during |
| | the GLP toxicology studies, additional safety assessments, including detailed |
| | monitoring for sensory changes as well as nerve conduction studies to be added to |
| | the clinical trial protocol. |
| | The Investigator has primary responsibility for the ongoing medical review of safety |
| | data (AEs, SAEs, laboratory data, etc.) throughout the study and prior to enrollment |
| | of each subject during the dose escalation phase. A Safety Review Board reviews |
| | safety data at specified intervals throughout the study and make recommendations to |
| | the Sponsor regarding further conduct of the study. The full details of the safety |
| | review process are still under discussion, but broadly speaking, safety evaluations |
| | after the first three subjects in Cohort 1 and after the first three subjects in Cohort 2 |
| | are conducted as described. A full list of events that are considered SRTs are |
| | included in the FIH study protocol. However, an outline of criteria/events that |
| | would result in study termination (i.e., no additional subjects would be enrolled, but |
| | all subjects who had already received rAAVhu68.hFXN would continue to receive |
| | follow-up for the planned duration of the study) or study suspension (i.e., |
| | enrollment would be temporarily halted to allow full review of the safety data) is |
| | included ). |
| | Decision tree for safety evaluations for the proposed Phase 1/2 trial is used in |
| | consideration for medical review. Medical review is performed by the Medical |
| | Monitor in conjunction with the Principal Investigator |
| | Abbreviations: AE, adverse event. |
| | Stopping Rules Include: |
| | Events that meet study stopping criteria include: |
| | A Grade 4 or 5 AE according to CTCAE version 5.0 criteria that the |
| | Investigator considers to be related to either the investigational product, or |
| | the IV administration or IDN injection procedure |
| | ALT or AST ≥3 × ULN and total bilirubin ≥2 × ULN and no other reason |
| | can be found to explain the changes observed |
| | The Safety Board reviews these AEs and renders a decision regarding continued |
| | conduct of the study and subject enrollment. |
| AAV- | Recombinant AAV vector genomes display inefficient integration into the host |
| associated | chromosome and predominantly persist in episomal form (McCarty et al., 2004). |
| Risks | The risk of tumorigenesis in humans due to insertional mutagenesis is unknown, but |
| | considered low at this time. |
| | NHP studies of ICM AAVhu68 delivery have demonstrated minimal to mild |
| | degeneration of peripheral sensory neurons in some animals. These lesions have not |
| | been associated with sensory deficits detectable by routine observations or on |
| | neurological exams. The risk of clinically meaningful sensory loss is therefore |
| | anticipated to be low. Detailed examinations are performed to evaluate sensory |
| | nerve toxicity, and sensory nerve conduction studies are employed in this trial to |
| | monitor for subclinical sensory neuron lesions. The 30 day observation period |
| | between subjects is expected to be sufficient to detect the development of sensory |
| | deficits, as sensory neuron lesions appear within 2-4 weeks after AAV |
| | administration in non-clinical studies. |
| | To assess potential immunogenic responses, the presence, absence, and titer of anti- |
| | AAVhu68 NAbs in both CSF and serum are examined at defined follow-up time |
| | points. |
| Statistical | No statistical comparisons are planned for safety evaluations. All results are |
| Methods | descriptive only. Data is listed, and summary tables are produced. |
| | Measurements at each time point are compared to baseline values for each subject, |
| | as well as natural history data from FRDA patients with comparable cohort |
| | characteristics where available for each endpoint. |
| | A statistical analysis plan is developed that describes the integration of the |
| | comparator dataset with the FIH trial data and details the planned analyses and |
| | comparisons. |

Subjects are admitted to the hospital for on the morning of Day-1 and remain in-hospital through Day 1, at least 24-hours post-IDN procedure to observe for any acute adverse events. At the discretion of the PI, and dependent upon a favorable safety evaluation, subjects may be discharged from the hospital and all subsequent visits performed as an outpatient. At the discretion of the principal investigator, subjects may remain in the hospital through Day 7, if the visits for this time period cannot be performed on an outpatient basis, as there are several visits during this period. Vector administration occurs in two processes: vector is first administered via IV infusion to a peripheral vein. After the IV infusion is completed, vector is then administered by direct injection using the ClearPoint® injection system. The intraparenchymal (dentate nucleus) injection should occur within 24 hours to prevent an immunologic reaction to the vector administered intravenously. Other laboratory assessments may be conducted as needed. Fasting is preferred but not required. Urine pregnancy testing is performed for women of child bearing potential only. A serum pregnancy test is performed in the event of a positive or equivocal urine pregnancy test result. Vital signs are monitored frequently throughout Days 0 and 1, including assessments every hour (+/−5 minutes) for the first 12 hours and every 2 hours for the following 36 hours, throughout the first 48 hours. During visits when an ECG is performed and vital signs are measured, the ECG should be performed first. On days where speech assessment is performed, this assessment should be performed first. Patients are administered corticosteroids immediately prior to and for 1 week post-IDN administration to minimize any potential brain inflammation related to the study procedure. The dosing regimen is tapered over the seven days of administration. If at any time post-IV/IDN administration transaminase elevations or hepatoxicity are observed, the steroid dose is increased or reinitiated. A full dosing regimen and frequency of administration, as well as a monitoring plan for risks associated with prophylactic corticosteroid use, is detailed in a protocol for the first-in-human study (above).

### C. Study Population Rationale

### Study Population Characteristics

The FIH trial focuses on patients ages ≥16 years diagnosed with early-onset (defined as age of onset <14 years) FRDA. This population was chosen for a FIH trial as they present with both the neurological and cardiac manifestations of the disease, progress at a faster rate, and are more homogeneous in their disease presentation than late-onset patients, making them the most appropriate population for whom a stabilizing, disease-modifying therapy would be most beneficial. These subjects also represent a population with high unmet need because disease-modifying or -stabilizing therapies for FRDA are still lacking.

The early-onset form of FRDA has a variable age of onset occurring between 10.5-15.5 years old (Harding, 1981; Filla et al., 1990; Dürr et al., 1996; Parkinson et al., 2013). The age of disease onset is correlated to severity of disease, with younger patients generally experiencing more severe symptoms and a faster rate of disease progression (Reetz et al., 2015). Conversely, late-onset FRDA patients typically display a more mild range of symptoms, with some never displaying the cardiac symptoms that are the most common cause of mortality in the general population of FRDA (De Michele et al., 1994; Bhidayasiri et al., 2005). Thus, as late-onset FRDA patients have a more variable distribution of symptoms and age of disease onset, designing a clinical trial that would unequivocally demonstrate efficacy of rAAVhu68.hFXN that includes these patients would be prohibitively difficult. Additionally, demonstration of improvement or stabilization of the late-onset form of the disease would not necessarily predict a demonstrable improvement in the more severe, early-onset, patients. Therefore, despite a shared underlying pathology between the early- and late-onset forms of the disease, we propose enrolling early-onset FRDA patients to test the efficacy of this gene product in the proposed FIH study. In certain embodiments, the population for whom this therapy is most appropriate are re-evaluated for subsequent studies.

### Justification of Neurologic and Cardiac Eligibility Criteria

The dose escalation phase (Stage 1) of the FIH study is intended to evaluate the safety of rAAVhu68.hFXN and identify a safe dose to take into further development, and given the lack of human experience with rAAVhu68.hFXN. The Stage 1 (Cohorts 1 and 2) enroll subjects with more advanced disease who have the highest unmet need for new therapies. Subjects recruited in Cohorts 1 and 2 are non-ambulatory, defined as scoring 5.0 or 5.5 out of 6 on the Functional Staging of Ataxia assessment which is validated for assessing ambulation for FRDA (Subramony et al., 2005). A score of 5.0 or 5.5 on this assessment recruits subjects who are non-ambulatory, but who do not have total dependency for all activities of daily living. As preservation and quality of speech are important to patients with FRDA, the potential impact on dysarthria from the IDN injection is assessed in this cohort. Therefore, the spontaneous speech subscore of the mFARS examination is used to recruit subjects for this study and enroll subjects who score a 0, 1 or 2 (out of 3) on this assessment at baseline. This score ensures the recruitment of subjects with dysarthria but who also have some preservation of speech.

Stage 2 (Cohort 3) enrolls subjects with less progressed disease who are ambulatory. Ambulatory is defined as being able to complete a 25-foot walk test in <1 minute with or without assistive devices and meeting the standing with eyes open criterion of the upright stability subsection the mFARS (i.e. score of 0 or ‘normal’ out of 4 points) which corresponds to the subject standing with their feet 20 cm apart and eyes open for >1 minute (Subramony et al., 2005; Rummey et al., 2020a). The goal of these inclusion criteria is to select a population in whom GTP-212 has the potential to improve or stabilize ambulation.

Patients with early-onset FRDA are predicted to completely lose ambulation 11.5 years after disease onset. According to this data, loss of ambulation occurs in a stepwise fashion. First, patients lose the ability to stand on their dominant foot, followed by the ability to stand in tandem, and subsequently the ability to stand feet together and eyes closed, typically all before receiving an FRDA diagnosis. After being diagnosed with FRDA, various additional aspects of ambulation are lost, with patients next losing the ability to stand with eyes closed and feet apart, followed by standing with eyes open and feet together, and finally with eyes open and feet apart. The first of these steps is predicted to occur an average of 4.1 years after diagnosis, followed by averages of 5.8 years and 9.3 years, respectively (Rummey et al., 2020a; Rummey et al., 2020b). Once these milestones are lost in the progression of the disease, they are not regained, thus stabilizing these patients as soon as possible after their diagnosis is critical to the maintenance of their ambulatory capabilities. As subjects recruited into this trial would likely have already lost some of these milestones at the trial start, the amount of time they are expected to remain ambulatory is less than the 11.5 years predicted time to ambulation loss beginning at the onset of disease. Furthermore, as there are no currently available treatments for FRDA (Section 3.4), treatment with rAAVhu68.hFXN would provide a potentially therapeutic option to these subjects that could allow for them to maintain or improve upon their current state of ambulation.

Additionally, it is possible that early treatment may result in stabilization or improvements in cardiac and neurological parameters of the disease. Requiring an mFARS score of >30 points allows for the recruitment of subjects who are in the early stages of disease progression and for whom stabilization or improvements in ambulation could be observed. Furthermore, this mFARS requirement prevents the recruitment of subjects who are asymptomatic or who have not yet progressed significantly in their disease. Based on feedback from key opinion leaders and physicians in the Friedreich's Ataxia field, an mFARS score of >30 allows us to recruit patients who can benefit the most from this treatment and allows us to monitor whether this therapy may affect neurological symptoms of the disease.

Although FRDA presents initially with ataxia symptoms, nearly all patients eventually develop cardiac symptoms later in disease progression. Since cardiac failure is the most common cause of death for FRDA patients (Tsou et al., 2011), one goal of this therapy is to prevent the manifestation of these cardiac symptoms. For this reason, subjects who are at a higher risk for future cardiac manifestations are recruited based on their baseline structural cardiac parameters. In Stage 1, subjects are required to meet one of the following structural cardiac parameters at baseline, as assessed by ECHO or cMRI readings: left-ventricular end diastolic diameter (LVEDD)>ULN, septal wall thickness (SWT)>ULN, or LV mass >ULN. These requirements were derived from the FA-COMS natural history data and key opinion leaders and physicians in the FRDA field as being sufficient to recruit patients with a higher than average risk of future cardiac manifestations. In Stage 2, subjects have less stringent cardiac inclusion criteria as they are earlier in disease progression. Specifically, subjects are required to have a septal wall thickness or LV mass above the upper limit of normal. By recruiting subjects with these parameters, we enroll patients at a high risk of developing cardiac symptoms within the timeframe of this clinical trial who would have the highest benefit from treatment with this therapy.

### Dual Route of Administration

A dual ROA allows to target both the cardiac and neurological manifestations of the disorder. Delivery of rAAVhu68.hFXN by an IV infusion as well as an IDN injection into each of the dentate nuclei is proposed as a mechanism by which to treat peripheral manifestations of the disease as well as the neurological aspects. Patients may receive this treatment via IV administration to prevent the manifestation of cardiac symptoms of this disease, including cardiac death. Delivery of rAAVhu68.hFXN via IDN administration also addresses the neurological manifestations of the disorder, such as the ataxia, dysmetria, and dysarthria, along with peripheral neuropathy observed in FRDA patients. Thus, delivering the vector via a dual administration to these tissues to allow for expression of frataxin is intended to prevent the more severe cardiac manifestations of the disease as well as stabilize or improve the ataxic symptoms of the disease. Details of the administration procedure for the IV administration and for the IDN administration are provided in Examples 7E, 7F, and 7G.

### Endpoints

In addition to measuring safety and tolerability as the primary endpoint, pharmacodynamic and efficacy endpoints were chosen on their ability to measure meaningful functional and clinical outcomes in this population. All neurological endpoints, with the exception of assessing frataxin expression in serum at 1 year post-rAAVhu68.hFXN treatment, are assessed continually throughout the trial and evaluated at 2 years post-dose. During the long-term follow up phase occurring for the last 3 years of the study, study visit frequency decrease to once every 6 months, with alternating evaluation of cardiac and neurological endpoints such that each set of measurements is evaluated annually. This approach allows for thorough evaluation of pharmacodynamics and clinical efficacy measures in treated subjects over a period of follow up for which untreated comparator data exist. Subjects continue to be monitored for safety and efficacy for a total of 5 years after rAAVhu68.hFXN administration, in accordance with “FDA Guidance for Industry: Long Term Follow-Up After Administration of Human Gene Therapy Products” (January 2020).

Following study completion, patients may be invited to enroll in a patient-registry for continued monitoring. The neurological symptoms of FRDA generally appear first with cardiac manifestations occurring later in disease progression. In focusing on the high-risk FRDA population, the goal is to recruit subjects who would be expected to show both neurologic and cardiac symptoms within trial time frame. Administration of rAAVhu68.hFXN is expected to improve disease status, thereby delaying the deterioration of neurological parameters and the onset of cardiac symptoms, both of which would be improvements in the disease for the target population. As the most common cause of mortality in these patients is cardiomyopathy (Tsou et al., 2011), rAAVhu68.hFXN is thereby expected to extend the life expectancy for these patients as well.

In line with how FDRA manifests, the proposed efficacy endpoints are divided into those that measure changes in the neurological and cardiac parameters of the disease. Changes in biomarkers and quality of life assessments are also summarized.

### Pharmacodynamic Endpoints

### Biomarkers

To assess expression of frataxin by rAAVhu68.hFXN, frataxin protein levels are measured in serum to monitor the level of vector expression. Serum is analyzed for frataxin levels at the pre-dose, 3 months, and 1 year time points using a lateral flow immunoassay, with the endpoint being evaluations at the 1-year timepoint. This assay has been described previously (Willis et al., 2008; Deutsch et al., 2010).

### Neurological Endpoints

To assess the effect of rAAVhu68.hFXN on the neurological progression in FRDA, the following parameters are evaluated relative to both the subject's values at baseline and to cohort-matched natural history data over the course of the 5 year follow up of the study. An interim analysis of all neurological endpoints are performed from data collected over the first 2 years of follow up. A final evaluation of neurological endpoints is performed with data collected over the 5 year duration of the study.

mFARS

An overall measure of neurological function is the FARS rating scale for Friedreich's Ataxia. The FARS scale is an exam-based rating scale that assesses neurological function over 5 areas of disease involvement (bulbar, upper limb, lower limb, peripheral nervous system, and upright stability) (Subramony et al., 2005). When compared with other FRDA rating scales, such as the International Cooperative Ataxia Rating Scale (ICARS), it was found to have the greatest effect size and require fewer patients and was therefore recommended for use in clinical trials (Fahey et al., 2007a). In addition to the FARS scoring system, there is a modified scale that uses only the subgroups of FARS involving the functional abilities of the patient (the bulbar, upper limb, lower limb, and upright stability subcategories) (Patel et al., 2016). The mFARS rating scales are now widely used in FRDA studies, and has been shown to strengthen the overall construct of the rating system versus the original FARS system (Rummey et al., 2019). It is therefore proposed to measure absolute mFARS scores over the course of the trial and compare these scores to both baseline values and cohort-matched natural history data for all treated subjects to assess how this therapy improves overall neurological function.

### Fine Motor Skills Assessment

In addition to using the mFARS rating scale to evaluate the effect of rAAVhu68.hFXN on neurological manifestations, further assessment of neurological symptoms are done by using the 9-hole peg test (9HPT). The 9HPT assesses fine motor skills by timing how long it takes for a subject to add pegs to a pegboard and remove them twice with each hand, beginning with their dominant hand. This assessment is validated and has been used in numerous FRDA trials to assess upper limb ambulation (Friedman et al., 2010; Patel et al., 2016; Lynch et al., 2019a; Lynch et al., 2019b). As patients from Stage 1 of the proposed trial may not be able to complete the 9HPT in <5 minutes, subjects in this cohort may instead perform an assessment that models use of a spoon, including the subject grasping, scooping, and transferring the spoon to their mouth, which also assesses upper limb motility but is an easier task for more severely affected subjects to perform (Nguyen et al., 2020). The endpoint is evaluated as a change from baseline in the timing of this task.

### Ambulation

To assess the ability of rAAVhu68.hFXN to improve or stabilize ambulation in subjects with FRDA, this trial measures subject scores on the upright stability subsection of the mFARS assessment as a secondary key efficacy endpoint. The upright stability subset of the mFARS has been used to evaluate the progressive loss of ambulation in subjects with FRDA, as well as to predict the average time to loss of ambulation in early-, mid-, and late-onset FRDA disease types using natural history data (Rummey et al., 2020a; Rummey et al., 2020b). This subset evaluates subjects on many aspects of ambulation, including assessments for sitting, standing, and gait.

Given the recent publication using the FA-COMS natural history data to analyze upright stability and loss of ambulation, this endpoint would have available natural history to be used as a direct comparator. Furthermore, in a recent analysis of the test-retest ability of the mFARS assessment, the upright stability subset of assessments was shown to be the most reliable measure in terms of intra-patient consistency in the assessment over multiple time points (Rummey et al., 2020a; Rummey et al., 2020b). Additionally, as this measure is scored on a rubric with pre-established, discrete intervals, it is expected that in addition to the reliability of this assessment itself, this data also has less variability than walk tests, which, per key opinion leader advice, are more subjective in terms of both subject ability and interpretation of how the assessment is to be completed. Given the natural history data that exists for this endpoint, as well as its demonstrated ability to be reliably captured and accurately track changes in the loss of ambulation, this measure is the best assessment of ambulation over time and thus using this assessment as a secondary key efficacy endpoint in Stage 2 of this trial.

To further assess mobility and ambulation in subjects from Stage 2 of this trial, a 25-foot walk test is measured over time as an exploratory endpoint. This assessment measures the amount of time it takes for the subject to complete a 25-foot walk, is a well established measure of ambulation used frequently in FRDA, and has been demonstrated to model real world ambulation (Fahey et al., 2007b; Milne et al., 2014). This endpoint is evaluated in subjects enrolled in Stage 2 of this trial only, and tests the ability of rAAVhu68.hFXN to stabilize or improve ambulation in patients for whom are expected to show a decrease in ambulation with no treatment over the course of 2 years.

### Dysarthria

To assess dysarthria in subjects over time in all cohorts over the course of the trial, speech analysis software developed by Redenlab in Queensland, Australia is utilized. This analysis involves subjects producing monosyllables or repeated syllable sounds, as well as, reading pre-defined passages out loud using an app available on devices such as a phone or tablet. The speech recording can then be sent for analysis by Renenlab specialists for different aspects of speech such as prosodic features (variation of pitch and loudness, maintenance of loudness, phrase length, general rate, and stress), respiratory features, phonatory features, resonance, articulatory features, and intelligibility (Folker et al., 2010; Vogel et al., 2017).

Furthermore, speech samples are also analyzed by the Assessment of Intelligibility of Dysarthria Speech criteria, encompassing features such as sentence intelligibility, total words per minute, and intelligible words per minute (Folker et al., 2010).

### Cardiac Endpoints

rAAVhu68.hFXN is evaluated via multiple cardiac endpoints, which are measured both relative to subject baseline and compared to cohort-matched natural history data. These assessments are able to demonstrate the ability of rAAVhu68.hFXN to address the cardiac symptoms seen in FRDA patients.

The following parameters are followed to assess the effects of rAAVhu68.hFXN on cardiac symptoms over the 5 year follow up of the study. An interim analysis of all cardiac endpoints is performed from data collected over the first 2 years of post-treatment follow up. A final evaluation of neurological endpoints is performed with data collected over the 5 year duration of the study.

### Structural Endpoints

In order to monitor stabilization or improvement in structural cardiac parameters of the disease, echocardiograms are performed throughout the course of the trial. Additionally, cardiac MRI (cMRI) is performed at baseline and annually throughout the 5-year course of the trial to obtain more detailed structural information to assess efficacy of rAAvhu68.hFXN.

Echocardiograms and cMRI data is collected to assess the efficacy of rAAvhu68.hFXN in stabilizing or improving the cardiac symptoms of FRDA subjects. The first endpoint evaluates relative wall thickness (RWT), defined here as 2 times posterior wall thickness divided by the LV end-diastolic internal diameter (LVEDID), for each patient. Although many FRDA patients display concentric thickening of ventricles, studies of echocardiograms from FRDA patients demonstrate that increases in RWT were among the most common LV abnormalities in this patient population (St John Sutton et al., 2014; Peverill et al., 2019). This endpoint is assessed by data from echocardiograms and cMRIs at 2 years in all cohorts.

Another structural endpoint that shows cardiac abnormalities early in FRDA progression is longitudinal strain. Longitudinal strain is defined as the change in the left ventricular segment length divided by the resting segment length obtained at mid-cavity level. A study by St. John Sutton and colleagues found that longitudinal strain in FRDA patients was significantly decreased from that of non-FRDA control patients. This study also found that decrease in strain was constant over time in the FRDA patients, and was unchanged over the course of 3 years relative to baseline values (St John Sutton et al., 2014). Data collected at the 2 year time point and throughout the course of the trial is analyzed to evaluate whether longitudinal strain is changed with rAAvhu68.hFXN treatment.

In addition to measuring RWT and longitudinal strain, echocardiogram and cMRI data is used to evaluate changes in left ventricular ejection fraction (LVEF) over the course of the trial as an exploratory endpoint. While a decline in LVEF is a late indicator of disease burden, it has been demonstrated in longitudinal natural history data that there is a subset of patients at a high risk of cardiovascular events for which LVEF would decline over the planned duration of this study (Pousset et al., 2015b). Considering that the first two cohorts of this trial consist of FRDA patients at the highest risk of cardiac complications, LVEF is monitored over the course of the trial to see if treatment with rAAvhu68.hFXN prevents subjects from showing a decline in this parameter. This endpoint is evaluated at 2 years and calculated for all echocardiogram and cMRI measurements taken from all cohorts.

### Electrocardiogram Endpoints

Electrocardiogram addition to the previously mentioned structural endpoint measures to further evaluate the efficacy of rAAvhu68.hFXN. Electrocardiograms (ECGs) for each subject are evaluated at each time point. While evaluating 12-lead ECGs is necessary for ongoing evaluation of the safety of rAAvhu68.hFXN, this data is also used to evaluate efficacy of rAAvhu68.hFXN. Specifically, changes in heart rate, R-R interval, PR interval, QRS interval, QT time and time corrected by Fridericia's formula (QTcF) are monitored. ECGs are monitored for abnormal findings, including non-specific ST-T wave changes, right axis deviation, left ventricular hypertrophy, right ventricular hypertrophy, as abnormalities in these ECG parameters have been observed in FRDA cohorts (Schadt et al., 2012). Furthermore, subjects are monitored for ventricular or supraventricular arrhythmias for all ECGs collected as proposed in the staging of cardiomyopathy criteria by (Weidemann et al., 2012). These parameters are monitored throughout the course of the trial and evaluated as an endpoint at the 2 year time point.

### Other Cardiac Endpoints

To further assess efficacy of rAAvhu68.hFXN on cardiac parameters, monitoring for the absence of progression of cardiac symptoms, including ICD, heart failure hospitalization, and survival is performed continuously throughout the trial and reported at the conclusion of the study.

### Quality of Life

In order to evaluate the ability of rAAvhu68.hFXN treatment to demonstrate an improvement in the quality of life of FRDA patients, the following quality of life assessments are evaluated as additional exploratory endpoints in all cohorts.

### FARA PRO

An FRDA-specific patient-reported outcomes (PRO) measure is utilized to evaluate quality of life as an additional exploratory endpoint. This PRO is currently in development by the Friedreich's Ataxia Research Alliance (FARA) and is expected to be validated by the start of the proposed trial. In utilizing a measure that is specific to the concerns of FRDA patients as an endpoint in the proposed study, a more accurate measure in the improvements made to the lives of these patients can be obtained.

### D. Intravenous Administration (FIH)

rAAvhu68.hFXN is administered via an IV infusion into a peripheral vein. The IV infusion rate is determined in the NHP nonclinical studies. For example, the rAAvhu68.hFXN is infused over no less than a 20-minute interval using a syringe infusion pump via an IV administration set. The interval can be prolonged to as much as 1 hour or longer if the investigator feels it is necessary to use a lower infusion rate. The IV infusion is performed first to allow for the observation of any hypersensitivity reactions to the gene product as well as other safety observations. This IV infusion occurs no longer than 24 hours prior to the IDN procedure occurring the following day.

Compatibility testing with the administration set and rAAvhu68.hFXN is performed. Variability in dosing levels can be caused by loss of the FDP through binding to plastics and other solid surfaces during vector storage and patient administration. Therefore, the clinically suitable surfactant Poloxamer 188 is a component of the final formulation buffer of the final rAAVhu68.hFXN formulation and is anticipated to minimize this type of loss. The interaction of the prepared final rAAVhu68.hFXN formulation with both the storage vial and the clinical IV and IDN devices is investigated to determine the amount of vector loss through binding to surfaces. For each of the delivery devices doses which bracket the anticipated doses for the clinical trial is prepared using an equivalent preparation process as used in the clinical trial. The prepared final rAAVhu68.hFXN formulation is passed through the each of the devices. GC titrations and potency assays are performed on pre-device and post-device samples. The appropriate number of replicates are included to assure statistical significance. Comparison of GC titers and potency pre- and post-device enables an assessment of final rAAVhu68.hFXN formulation loss administration to the patient. Parallel studies are also be performed in a similar way to assess the in-use stability rAAVhu68.hFXN formulation after preparation and storage in the delivery syringe.

### E. Intraparenchymal (Dentate Nucleus) Injection Device

The devices that is used for the IDN injection are the ClearPoint® System and Accessories and Ventricular Cannula.

### F. Dosing Regimen and Explanation of the Device Use by the Clinician

Following the IV infusion of rAAVhu68.hFXN, an IDN injection into each of the dentate nuclei of the cerebellum is performed as this is a major site of neurological pathology in FRDA. The direct injection procedure is performed early in the morning after the subject receives a dose by IV infusion the previous day using the ClearPoint® injection system. The ClearPoint® injection system consists of a monitor to visualize the brain and injection procedure in real time, a head fixation frame that is secured to the skull, and an MRI-compatible SmartFrame® trajectory device that enables MRI-guided alignment during the procedure. This system allows for the direct injection to be combined with real-time visualization of the injection tract by MRI. To enable visualization of vector distribution, the injection material containing the vector is be mixed with gadolinium (final concentration of 2 mM gadolinium). Proper precautions are taken with the gadolinium, including warning patients of the potential risks of gadolinium use and prolonged gadolinium retention for brain MRI in informed consent forms. Furthermore, patients for whom there are increased safety concerns with gadolinium use, such as women who are pregnant or those with kidney disease, are already excluded from participation in this trial.

During the direct injection procedure, the injection cannula is placed through the ClearPoint® frame to the correct position on the skull and the frame maintains the correct trajectory. The final position of the injection cannula is confirmed using real-time MRI images, and then the vector is injected into the parenchyma of the deep cerebellar nuclei using convection-enhanced delivery. Each subject receives administration of the rAAVhu68.hFXN plus gadolinium in each dentate nucleus injected at a rate of 0.5 L/min initially, and then at an increased rate of up to 5 L/min based on clinician discretion during the procedure. The procedure takes approximately 5-6 hours and the subjects are anesthetized for the duration of the procedure.

All documents cited in this specification are incorporated herein by reference, U.S. patent application Ser. No. 17/787,013, filed Jun. 17, 2022, International Patent Application No. PCT/US2020/066167, filed Dec. 19, 2020, as is the priority application, U.S. Provisional Patent Application No. 62/950,834, filed Dec. 19, 2019. The Sequence Listing filed herewith, labeled 20-9217PCT_ST25.txt, and the sequences and text therein are incorporated by reference. While the invention has been described with reference to particular embodiments, it will be appreciated that modifications can be made without departing from the spirit of the invention. Such modifications are intended to fall within the scope of the appended claims.

### (Sequence Listing Free Text)

The following information is provided for sequences containing free text under numeric identifier <223>.

| SEQ ID NO: | |
|---|---|
| (containing | |
| free text) | Free text under <223> |
| 3 | <223> Engineered hFXN coding |
| | sequence |
| 8 | <223> CB7.CI.hFXNco |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> TATA_signal |
| | <222> (836) . . . (838) |
| | <220> |
| | <221> Intron |
| | <222> (958) . . . (1930) |
| | <220> |
| | <221> misc_feature |
| | <222> (1942) . . . (2572) |
| | <223> human FXN |
| | <220> |
| | <221> polyA_signal |
| | <222> (2611) . . . (2737) |
| | <223> Rabbit globin polyA |
| | <220> |
| | <221> repeat_unit |
| | <222> (2826) . . . (2955) |
| | <223> 3′ ITR |
| 11 | <223> AAVrh.91 |
| 12 | <223> vector genome |
| | AAV.CB7.CI.hFXNco.RBG |
| | <220> |
| | <221> repeat_region |
| | <222> (1) . . . (130) |
| | <223> 5′ITR |
| | <220> |
| | <221> repeat_region |
| | <222> (198) . . . (579) |
| | <223> CMV IE enhancer |
| | <220> |
| | <221> promoter |
| | <222> (585) . . . (862) |
| | <223> Chicken beta-actin promoter |
| | <220> |
| | <221> TATA_signal |
| | <222> (836) . . . (862) |
| | <223> TATA |
| | <220> |
| | <221> Intron |
| | <222> (957) . . . (1929) |
| | <223> chicken beta-actin intron |
| | <220> |
| | <221> Intron |
| | <222> (957) . . . (1929) |
| | <223> chimeric intron |
| | <220> |
| | <221> misc_feature |
| | <222> (1941) . . . (2570) |
| | <223> hFXNco |
| | <220> |
| | <221> polyA_signal |
| | <222> (2610) . . . (2736) |
| | <223> Rabbit globin poly A |
| | <220> |
| | <221> repeat_region |
| | <222> (2825) . . . (2954) |
| | <223> 3′ITR |

1. A recombinant adeno-associated virus (rAAV) having an AAV capsid and a vector genome comprising a FXN gene having the sequence of SEQ ID NO: 3 or a sequence 95% identical thereto that encodes human frataxin, and regulatory sequences which direct expression of the FXN gene in targeted human cells.

2-51. (canceled)


SEQUENCE LISTING (12 sequences)

SEQ ID NO: 1 - DNA, 630 bp, Homo sapiens
  source 1..630: mol_type = other DNA
  CDS 1..630
atgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgct

SEQ ID NO: 2 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - DNA, 630 bp, synthetic construct
  misc_feature 1..630: note = Engineered hFXN coding sequence
  source 1..630: mol_type = other DNA
atgtggacacttggccgcagagctgttgctggcctgcttgcttctccatctcccgctcaagcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggacgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatacgattggaccggcaagaactgggtgtacagccacgacggcgtgtccctgcacgaactgctggctgccgaactgacaaaggccctgaaaacaaagctggacctgtccagcctggcctactctggcaaggatgcc

SEQ ID NO: 4 - DNA, 2211 bp, Adeno-associated virus
  source 1..2211: mol_type = other DNA; serotype = 68
  CDS 1..2211
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 5 - protein, 736 aa, Adeno-associated virus
  source 1..736: note = serotype 68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - DNA, 2208 bp, Adeno-associated virus
  source 1..2208: mol_type = other DNA; serotype = 9
  CDS 1..2208: note = AAV9 VP1 Capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 7 - protein, 736 aa, Adeno-associated virus
  source 1..736: note = serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 2955 bp, synthetic construct
  misc_feature 1..2955: note = CB7.CI.hFXNco
  repeat_region 1..130: note = 5' ITR
  regulatory 198..579: note = promoter - CMV IE promoter; regulatory_class = promoter
  regulatory 582..863: note = promoter - CB promoter; regulatory_class = promoter
  regulatory 836..838: regulatory_class = TATA_box
  misc_feature 958..1930
  misc_feature 1942..2572: note = human FXN
  regulatory 2611..2737: note = polyA_signal - Rabbit globin polyA; regulatory_class = polyA_signal_sequence
  repeat_region 2826..2955: note = repeat_unit - 3' ITR; rpt_unit_range = 2826..2955
  source 1..2955: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtggacacttggccgcagagctgttgctggcctgcttgcttctccatctcccgctcaagcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggacgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatacgattggaccggcaagaactgggtgtacagccacgacggcgtgtccctgcacgaactgctggctgccgaactgacaaaggccctgaaaacaaagctggacctgtccagcctggcctactctggcaaggatgcctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 2211 bp, Adeno-associated virus
  source 1..2211: mol_type = other DNA; serotype = rh.91
  CDS 1..2211
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 10 - protein, 736 aa, Adeno-associated virus
  source 1..736: note = serotype rh.91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - DNA, 2211 bp, Adeno-associated virus
  misc_feature 1..2211: note = AAVrh.91
  source 1..2211: mol_type = other DNA; serotype = rh.91
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 12 - DNA, 2954 bp, synthetic construct
  misc_feature 1..2954: note = vecotr genome AAV.CB7.CI.hFXNco.RBG
  repeat_region 1..130: note = 5'ITR
  repeat_region 198..579: note = CMV IE enhancer
  regulatory 585..862: note = promoter - Chicken beta-actin promoter; regulatory_class = promoter
  regulatory 836..862: note = TATA_signal - TATA; regulatory_class = TATA_box
  misc_feature 957..1929: note = Intron - chicken beta-actin intron
  misc_feature 957..1929: note = Intron - chimeric intron
  misc_feature 1941..2570: note = hFXNco
  regulatory 2610..2736: note = polyA_signal - Rabbit globin poly A; regulatory_class = polyA_signal_sequence
  repeat_region 2825..2954: note = 3'ITR
  source 1..2954: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtggacacttggccgcagagctgttgctggcctgcttgcttctccatctcccgctcaagcccagacactgaccagagtgcctagacctgctgaactggcccctctgtgtggcagaagaggcctgagaaccgacatcgacgccacatgcacacctagaagggccagcagcaatcagcggggcctgaatcagatctggaacgtgaagaaacagagcgtgtacctgatgaacctgagaaagagcggcaccctgggacaccctggaagcctggacgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatacgattggaccggcaagaactgggtgtacagccacgacggcgtgtccctgcacgaactgctggctgccgaactgacaaaggccctgaaaacaaagctggacctgtccagcctggcctactctggcaaggatgcctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag